(19) United States
(12) Patent Application Publication
(10) Pub. No.: US 2023/0233583 A1
(43) Pub. Date: Jul. 27, 2023

(54) USE OF PHYTOCANNABINOIDS FOR TREATING MULTIPLE MYELOMA

(71) Applicant: Entourage Biosciences Inc., Vancouver (CA)

(72) Inventors: Massimo Nabissi, San Ginesio (IT); Oliviero Marinelli, Colonnella (IT); Cristina Aguzzi, Falconara M.Ma (IT); Laura Zeppa, Camerino (IT); Sazzad Hossain, Richmond (CA)

(21) Appl. No.: 18/009,688

(22) PCT Filed: Jun. 11, 2021
(86) PCT No.: PCT/CA2021/050801

§ 371 Date: Dec. 9, 2022

Related U.S. Application Data
(60) Provisional application No. 63/038,532, filed on Jun. 12, 2020.

(51) Int. Cl. A61K 31/00; A61K 45/06; A61P 35/00

(52) U.S. Cl. CPC A61K 31/658; A61K 45/06; A61P 35/00

(57) ABSTRACT

The present disclosure relates to the use of suitable phytocannabinoids such as cannabidivarin, cannabinol, cannabigerol or cannabichromene or combinations thereof for treatment of multiple myeloma and/or similar conditions, diseases or disorders.

### CROSS-REFERENCE TO RELATED APPLICATIONS

The present application claims the benefit of priority from co-pending U.S. provisional application No. 63/038,532 filed on Jun. 12, 2020, the contents of which are incorporated herein by reference in their entirety.

### FIELD

The present disclosure relates to the use of phytocannabinoids for treatment of plasma cell neoplasms, for example, the use of cannabidivarin, cannabinol, cannabigerol, cannabichromene or combinations thereof for treatment of multiple myeloma.

### BACKGROUND

Multiple myeloma (MM) is the second most frequent haematological malignancy. Therapeutic approaches such as immune-modulatory drugs and proteasome inhibitors, have allowed progress in the control of MM.

Bone disease affects up to 80% of the newly diagnosed patients with multiple myeloma (NDMM). These patients present with substantial bone pain, and they are at high risk for developing skeletal-related events (SREs), such as pathological fractures or spinal cord compression.¹ Myeloma bone disease is characterized by significant increased osteoclast activity and suppressed osteoblast function, leading to bone loss.²

US Patent Application Publication No. 2016/0120874 generally discloses the use of cannabinoid agents for preparing a medicinal product for the treatment of monoclonal gammopathies such as MM, but the experiments disclosed in the application only tested WIN 55-212.2. US Patent Application Publication No. 2018/0353461 generally discloses the use of a ceramide-generating anticancer agent or treatment and/or a ceramide degradation inhibitor for use in the prevention, treatment, or amelioration of cancer or tumors of the hematopoietic and lymphoid tissues, but the experiments disclosed in the application only tested WIN-55,212-2 mesylate and JWH-133. PCT Application Publication No. 2019/222459 generally discloses cannabinoid preparations for use in cancer treatments such as for MM. The cannabinoid preparations of WO 2019/222459 contain at least 99% by weight cannabidiol (CBD).

Studies have showed a potential anti-tumor role for certain cannabinoids, by means of modulating cell signaling pathways involved, for example, in cancer cell proliferation, chemo-resistance and/or migration. For example, it has been reported that CBD by itself or in synergy with bortezomib (BORT) strongly inhibited growth, arrested cell cycle progression and induced MM cells death by regulating the ERK, AKT and NF-κB pathways³. Additionally, it has been reported that the combination of CBD with Δ⁹-tetrahydrocannabinol (THC) was able to reduce MM cells migration by down-regulating expression of the chemokine receptor CXCR4 and of the CD147 plasma membrane glycoprotein and also that this combination acts in synergy with carfilzomib (CFZ) to increase MM cell death and inhibit cell migration⁴.

However, MM is still considered incurable and the discovery of new treatments to improve the currently available therapies remains desirable.

### SUMMARY

The phytocannabinoids cannabigerol (CBG), cannabinol (CBN), cannabichromene (CBC) and cannabidivarin (CBDV) alone, and in combinations, induced cytotoxicity in multiple myeloma (MM) cell lines. A dose dependent effect in all MM cell lines was observed for all individual phytocannabinoids with the efficacy in reducing cell viability generally highest for CBDV, followed by CBN, CBG and CBC. The combination of CBG with CBC was observed to have the highest efficacy in reducing cell viability followed by combinations comprising CBN and one of the other phytocannabinoids. To evaluate the cytotoxic mechanism, MM cell lines were treated daily with a cytotoxic dose for each phytocannabinoid and the results suggested that all induced necrotic cell death. This was confirmed via Western blot and Comet assay. An invasion assay in a co-culture of HuOB and MM cell lines RPMI and U266 was performed, which showed that in both MM cell lines, the phytocannabinoids exerted an inhibitory effect on MM cell invasion. A higher activity was observed for CBG and CBN, followed by CBC and CBDV. The combination of CBG plus CBN showed nearly complete inhibition of MM cell invasion.

Accordingly, the present disclosure includes a method of treating a plasma cell neoplasm in a subject in need thereof, comprising administering an effective amount of at least one phytocannabinoid to the subject, with the proviso that the at least one phytocannabinoid does not comprise cannabidiol or tetrahydrocannabinol. The present disclosure also includes a use of an effective amount of at least one phytocannabinoid for treatment of a plasma cell neoplasm in a subject in need thereof, with the proviso that the at least one phytocannabinoid does not comprise cannabidiol or tetrahydrocannabinol. The present disclosure also includes a use of an effective amount of at least one phytocannabinoid for preparation of a medicament for treatment of a plasma cell neoplasm in a subject in need thereof, with the proviso that the at least one phytocannabinoid does not comprise cannabidiol or tetrahydrocannabinol. The present disclosure also includes at least one phytocannabinoid for use to treat a plasma cell neoplasm in a subject in need thereof, with the proviso that the at least one phytocannabinoid does not comprise cannabidiol or tetrahydrocannabinol.

In an embodiment, the at least one phytocannabinoid comprises cannabidivarin, cannabinol, cannabigerol, cannabichromene or combinations thereof. In another embodiment of the present disclosure, the at least one phytocannabinoid is cannabidivarin, cannabinol, cannabigerol, cannabichromene or combinations thereof.

In an embodiment, the at least one phytocannabinoid is cannabidivarin. In another embodiment, the at least one phytocannabinoid is cannabinol. In another embodiment, the at least one phytocannabinoid is cannabichromene. In another embodiment, the at least one phytocannabinoid is cannabigerol. In another embodiment, the at least one phytocannabinoid is a combination of cannabigerol and cannabichromene.

In an embodiment, the combination of at least one phytocannabinoid that is cannabidivarin, cannabinol, cannabigerol or cannabichromene is a combination comprising cannabinol. In another embodiment, the combination further consists of cannabigerol. In a further embodiment, the combination further consists of cannabichromene. In another embodiment, the combination further consists of cannabidivarin.

In an embodiment, the plasma cell neoplasm is multiple myeloma.

In an embodiment, the subject has myeloma bone disease and the at least one phytocannabinoid is further for treatment of the myeloma bone disease.

The present disclosure also includes a method of treating myeloma bone disease in a subject in need thereof, comprising administering an effective amount of at least one phytocannabinoid to the subject, with the proviso that the at least one phytocannabinoid does not comprise cannabidiol or tetrahydrocannabinol. The present disclosure also includes a use of an effective amount of at least one phytocannabinoid for treatment of myeloma bone disease in a subject in need thereof, with the proviso that the at least one phytocannabinoid does not comprise cannabidiol or tetrahydrocannabinol. The present disclosure also includes a use of an effective amount of at least one phytocannabinoid for preparation of a medicament for treatment of myeloma bone disease in a subject in need thereof, with the proviso that the at least one phytocannabinoid does not comprise cannabidiol or tetrahydrocannabinol. The present disclosure also includes at least one phytocannabinoid for use to treat myeloma bone disease in a subject in need thereof, with the proviso that the at least one phytocannabinoid does not comprise cannabidiol or tetrahydrocannabinol.

In an embodiment, the at least one phytocannabinoid is cannabinol. In another embodiment, the at least one phytocannabinoid is cannabigerol. In another embodiment, the at least one phytocannabinoid is a combination of cannabinol and cannabigerol.

In an embodiment, the at least one phytocannabinoid is administered or for use in combination with at least one other anticancer treatment.

In an embodiment, the other anticancer treatment is a proteasome inhibitor, an immunomodulator or combinations thereof.

In an embodiment, the subject is a human.

In an embodiment, the at least one phytocannabinoid is administered to the subject in the form of a pharmaceutical composition comprising the at least one phytocannabinoid and a pharmaceutically acceptable carrier. In an embodiment, the at least one phytocannabinoid is administered orally to the subject. In another embodiment, the at least one phytocannabinoid is administered to the subject in the form of a pharmaceutical composition comprising the at least one phytocannabinoid and a pharmaceutically acceptable oil. In a further embodiment, the at least one phytocannabinoid is administered to the subject in the form of a gastro-resistant capsule comprising the at least one phytocannabinoid. In an alternative embodiment, the at least one phytocannabinoid is administered to the subject by inhalation via vaporization. In another embodiment, the at least one phytocannabinoid is administered to the subject once daily.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

The embodiments of the disclosure will now be described in greater detail with reference to the attached drawings, in which:

FIGS. 1-4 are plots of cell viability (% vs vehicle) as a function of dose (log μM) showing that phytocannabinoids cannabigerol (CBG; FIG. 1), cannabichromene (CBC; FIG. 2), cannabinol (CBN; FIG. 3) and cannabidivarin (CBDV; FIG. 4) induce cytotoxicity in MM cell lines U266, SKO and RPMI according to exemplary embodiments of the present disclosure. Cell lines were treated with daily administration of different doses of phytocannabinoid as indicated on the plots. Cell viability was evaluated at 72 hours post-treatment, by MTT assay. Data shown are expressed as mean±SE of two separate experiments and six wells for each dose. * p<0.05. In FIGS. 1-4 the IC₅₀ was calculated by PRISM 5 software.

FIGS. 5-16 are plots showing that phytocannabinoids CBG (FIGS. 5-7); CBC (FIGS. 8-10); CBN (FIGS. 11-13) and CBDV (FIGS. 14-16) induce necrosis in SKO (FIGS. 5, 8, 11 and 14); U266 (FIGS. 6, 9, 12 and 15); and RPMI (FIGS. 7, 10, 13 and 16) cell lines according to exemplary embodiments of the present disclosure. Cell lines were treated for 48 hours with CBG (25 μM), CBN (25 μM), CBDV (25 μM) or CBC (75 μM) and the percentage of Annexin-V/PI positive cells were determined by FACS analysis. Values in overlays on each plot: upper left=necrotic cells; upper right=late death cells (both apoptotic and necrotic); lower left=live cells; and lower right=apoptotic cells. Data for each phytocannabinoid (lower plots in each Figure) expressed as percentage of PI positive cells with respect to vehicle treated cells (upper plots in each Figure).

FIGS. 17-20 show phytocannabinoids increased H2AX levels in MM cell lines according to exemplary embodiments of the present disclosure. 4×10⁴ cells were plated in 6 well plates and treated for 48 hours with daily administration of CBG (25 μM; FIG. 17), CBC (75 μM; FIG. 18), CBN (25 μM; FIG. 19) or CBDV (25 μM; FIG. 20). Densitometric analysis was carried out by evaluating two independent experiments by a Chemidoc using the Quantity One software (BioRad, Hercules, USA). H2AX densitometry values were normalized to GAPDH used as loading control and normalized with non-treated cells. Densitometric values shown are the mean±SE of two separate experiments.

FIGS. 21-24 show phytocannabinoids induce DNA damage in MM cell lines according to exemplary embodiments of the present disclosure. Damage to cellular DNA of the cell lines SKO (upper images in each of FIGS. 21-24), U266 (middle images in each of FIGS. 21-24) and RPMI (lower images in each of FIGS. 21-24) due to exposure to CBG (25 μM; FIG. 21), CBC (75 μM; FIG. 22), CBN (25 μM; FIG. 23) and CBDV (25 μM; FIG. 24) were assessed using the Comet assay. The normal untreated cell lines are round-shaped (left images in each of FIGS. 21-24), while the ones treated with phytocannabinoids showed a slight tail (right images in each of FIGS. 21-24).

FIG. 25 is a plot showing cell viability (% vs vehicle) for phytocannabinoid combinations according to exemplary embodiments of the present disclosure in comparison to vehicle control (far right columns) for MM cell lines U266, RPMI and SKO.

FIG. 26 is a plot showing relative expression (vs GADPH) for, from left to right: human cannabinoid receptor 1 (CB1), human cannabinoid receptor 2 (CB2) and transient receptor potential (TRP) TRPV1, TRPV2, TRPV3, TRPV4, TRPM8 and TRPA1 target genes in MM cell lines U266, RPMI and SKO (from left to right for each target gene). Target gene mRNA expression was evaluated by qRT-PCR in U266, RPMI, and SKO cell lines. Target mRNA levels were normalized for GAPDH expression. Data are expressed as fold mean±SE.

FIG. 27 shows CB expression in MM cell lines U266 (left), RPMI (center) and SKO (right). CB1 and CB2 protein expression was evaluated by western blot in U266, RPMI and SKO cell lines. Densitometric values were normalized to GAPDH used as a loading control. Densitometric values shown are the mean±SE of three separate experiments.

FIG. 28 shows TRPV expression in MM cell lines U266 (left), RPMI (center) and SKO (right). TRPV1, TRPV2, TRPV3 and TRPV4 protein expression were evaluated by western blot in U266, RPMI and SKO cell lines. Densitometric values were normalized to GAPDH used as a loading control. Densitometric values shown are the mean±SE of three separate experiments.

FIG. 29 shows TRPM8 and TRPA1 expression in MM cell lines U266 (left), RPMI (center) and SKO (right). TRPM8 and TRPA1 protein expression were evaluated by western blot in U266, RPMI and SKO cell lines. Densitometric values were normalized to GAPDH used as a loading control. Densitometric values shown are the mean±SE of three separate experiments.

FIG. 30 shows cannabinoid target expression in MM cell lines. CB1, CB2, TRPV1, TRPV2, TRPV3, TRPV4 and TRPM8 protein expression were evaluated in U266, RPMI and SKO cell lines. Densitometric values were normalized to GAPDH used as a loading control. Densitometric values shown are the mean±SE of three separate experiments.

FIG. 31 shows the dose response of cannabinoids CBG (upper left), CBC (upper right), CBN (lower left) and CBDV (lower right) in the HuOB cell line. HuOB were treated with daily administration of different doses of phytocannabinoid as indicated on the plots and cell viability was analysed at 72 hours post-treatment, by MTT assay. Each compound was evaluated in six wells and in two separate experiments. The values are represented as % of cell viability compared to vehicle-treated cells. The standard deviation of the data reported was <10%.

FIG. 32 shows cannabinoid receptors CB1, CB2, TRPV1, TRPV2, TRPV3, TRPV4, TRPM8 and TRPA1 detected in HuOB by Western Blot analysis.

FIG. 33 shows a comparative analysis of cannabinoid target expression in HuOB and MM cell lines; from left to right for each cell line: CB1, CB2, TRPV1, TRPV2, TRPV3, TRPV4, TRPM8 and TRPA1.

FIG. 34 shows plots of cell invasion for RPMI (upper plot) and U266 (lower plot) MM cell lines for the indicated phytocannabinoid treatments in comparison to controls.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form thereof, such as “comprise” and “comprises”), “having” (and any form thereof, such as “have” and “has”), “including” (and any form thereof, such as “include” and “includes”) or “containing” (and any form thereof, such as “contain” and “contains”), are inclusive or open-ended and do not exclude additional, unrecited elements or process/method steps. As used herein, the word “consisting” and its derivatives are intended to be close-ended terms that specify the presence of the stated features, elements, components, groups, integers and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the term it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is present or used.

The term “cannabigerol” and the abbreviation “CBG” as used herein refer to the phytocannabinoid 2-[(2E)-3,7-dimethylocta-2,6-dienyl]-5-pentylbenzene-1,3-diol of the structure:

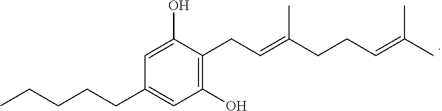 [SMILES: CCCCCc1cc(O)c(C/C=C(\C)CCC=C(C)C)c(O)c1]

The term “cannabinol” and the abbreviation “CBN” as used herein refer to the phytocannabinoid 6,6,9-trimethyl-3-pentylbenzo[c]chromen-1-ol of the structure:

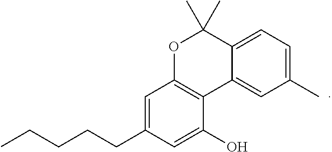 [SMILES: CCCCCc1cc(O)c2c(c1)OC(C)(C)c1ccc(C)cc1-2]

The term “cannabichromene” and the abbreviation “CBC” as used herein refer to the phytocannabinoid 2-methyl-2-(4-methylpent-3-enyl)-7-pentylchromen-5-ol of the structure:

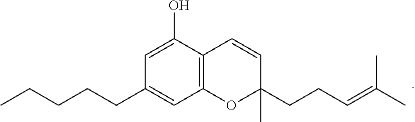 [SMILES: CCCCCc1cc(O)c2c(c1)OC(C)(CCC=C(C)C)C=C2]

The term “cannabidivarin” and the abbreviation “CBDV” as used herein refer to the phytocannabinoid 2-[(1R,6R)-3-methyl-6-prop-1-en-2-ylcyclohex-2-en-1-yl]-5-propylbenzene-1,3-diol of the structure:

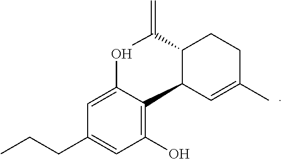 [SMILES: C=C1Oc2cc(CCC)cc(O)c2[C@@H]2C=C(C)CC[C@@H]12]

The term “plasma cell neoplasm” as used herein refers, for example, to a disease, condition or disorder in which there is clonal proliferation of immunoglobulin-secreting plasma cells. Plasma cell neoplasms are associated with M proteins (sometimes referred to as monoclonal or myeloma proteins). Plasma cell neoplasms include multiple myeloma, Waldenstrom's macroglobulinemia, monoclonal gammopathy of undetermined significance (MGUS), and plasmacytoma (isolated plasmacytoma of the bone or extramedullary plasmacytoma).

The term “subject” as used herein includes all members of the animal kingdom including mammals. In an embodiment, the subject is a human.

The term “pharmaceutically acceptable” means compatible with the treatment of subjects, for example, mammals such as humans.

The term “enteral” as used herein means taken into the body or administered or used in a manner that is through the gastrointestinal tract.

The term “parenteral” as used herein means taken into the body or administered or used in a manner other than through the gastrointestinal tract.

The terms “to treat”, “treating”, “treatment” and the like as used herein and as is well understood in the art, refer to an approach for obtaining beneficial or desired results, including clinical results. Beneficial or desired results include, but are not limited to, alleviation or amelioration of one or more symptoms of a disease, condition or disorder such as multiple myeloma, diminishment of the extent of the disease, condition or disorder such as multiple myeloma, stabilized (i.e. not worsening) disease, condition or disorder such as multiple myeloma, delay or slowing of the progression of the disease, condition or disorder such as multiple myeloma, amelioration or palliation of the state of the disease, condition or disorder such as multiple myeloma and/or remission (whether partial or total) of the disease, condition or disorder such as multiple myeloma, whether detectable or undetectable. “To treat”, “treating”, “treatment” and the like as used herein also include prophylactic treatment of the disease, condition or disorder such as multiple myeloma. For example, a subject with early stage multiple myeloma is treated to prevent or delay progression or alternatively a subject in remission is treated to prevent or delay recurrence.

As used herein, the term “effective amount” and the like means an amount effective, at dosages and for periods of time necessary to achieve a desired result. For example, in the context of treating multiple myeloma, an effective amount of the at least one phytocannabinoid is an amount that, for example, reduces the multiple myeloma compared to the multiple myeloma without administration of the at least one phytocannabinoid. By “reducing the multiple myeloma” it is meant, for example, reducing the number of myeloma cells, reducing the symptoms of the multiple myeloma and/or slowing the advancement of the multiple myeloma. Effective amounts may vary according to factors such as the disease state, age, sex and/or weight of the subject. The amount of the at least one phytocannabinoid that will correspond to such an amount will vary depending upon various factors, such as the given phytocannabinoid or combination thereof, the pharmaceutical formulation, the route of administration or use, the identity of the subject being treated, and the like, but can nevertheless be routinely determined by one skilled in the art having reference to this disclosure.

### II. Methods and Uses

The phytocannabinoids cannabigerol (CBG), cannabinol (CBN), cannabichromene (CBC) and cannabidivarin (CBDV) alone, and in combinations, induced cytotoxicity in multiple myeloma (MM) cell lines. To evaluate the cytotoxic mechanism, MM cell lines were treated daily with a cytotoxic dose for each phytocannabinoid and the results suggested that all induced necrotic cell death. This was confirmed via Western blot and Comet assay. An invasion assay in a co-culture of HuOB and MM cell lines RPMI and U266 was performed, which showed that in both MM cell lines, the phytocannabinoids exerted an inhibitory effect on MM cell invasion. A higher activity was observed for CBG and CBN, followed by CBC and CBDV. The combination of CBG plus CBN showed nearly complete inhibition of MM cell invasion.

Accordingly, the present disclosure includes a method of treating a plasma cell neoplasm in a subject in need thereof, comprising administering an effective amount of at least one phytocannabinoid to the subject, with the proviso that the at least one phytocannabinoid does not comprise cannabidiol or tetrahydrocannabinol. The present disclosure also includes a method of treating a plasma cell neoplasm in a subject in need thereof, comprising administering an effective amount of at least one phytocannabinoid to the subject, with the proviso that the at least one phytocannabinoid does not comprise cannabidiol. The present disclosure also includes a method of treating a plasma cell neoplasm in a subject in need thereof, comprising administering an effective amount of at least one phytocannabinoid to the subject, with the proviso that the at least one phytocannabinoid does not comprise tetrahydrocannabinol.

The present disclosure also includes a use of an effective amount of at least one phytocannabinoid for treatment of a plasma cell neoplasm in a subject in need thereof, with the proviso that the at least one phytocannabinoid does not comprise cannabidiol or tetrahydrocannabinol. The present disclosure also includes a use of an effective amount of at least one phytocannabinoid for treatment of a plasma cell neoplasm in a subject in need thereof, with the proviso that the at least one phytocannabinoid does not comprise cannabidiol. The present disclosure also includes a use of an effective amount of at least one phytocannabinoid for treatment of a plasma cell neoplasm in a subject in need thereof, with the proviso that the at least one phytocannabinoid does not comprise tetrahydrocannabinol.

The present disclosure also includes a use of an effective amount of at least one phytocannabinoid for preparation of a medicament for treatment of a plasma cell neoplasm in a subject in need thereof, with the proviso that the at least one phytocannabinoid does not comprise cannabidiol or tetrahydrocannabinol. The present disclosure also includes a use of an effective amount of at least one phytocannabinoid for preparation of a medicament for treatment of a plasma cell neoplasm in a subject in need thereof, with the proviso that the at least one phytocannabinoid does not comprise cannabidiol. The present disclosure also includes a use of an effective amount of at least one phytocannabinoid for preparation of a medicament for treatment of a plasma cell neoplasm in a subject in need thereof, with the proviso that the at least one phytocannabinoid does not comprise tetrahydrocannabinol.

The present disclosure also includes at least one phytocannabinoid for use to treat a plasma cell neoplasm in a subject in need thereof, with the proviso that the at least one phytocannabinoid does not comprise cannabidiol or tetrahydrocannabinol. The present disclosure also includes at least one phytocannabinoid for use to treat a plasma cell neoplasm in a subject in need thereof, with the proviso that the at least one phytocannabinoid does not comprise cannabidiol. The present disclosure also includes at least one phytocannabinoid for use to treat a plasma cell neoplasm in a subject in need thereof, with the proviso that the at least one phytocannabinoid does not comprise tetrahydrocannabinol.

In an embodiment, the at least one phytocannabinoid comprises, consists essentially of or consists of (or “is”) cannabidivarin, cannabinol, cannabigerol, cannabichromene or combinations thereof. In another embodiment, the at least one phytocannabinoid comprises cannabidivarin, cannabinol, cannabigerol, cannabichromene or combinations thereof. In a further embodiment, the at least one phytocannabinoid consists essentially of cannabidivarin, cannabinol, cannabigerol, cannabichromene or combinations thereof. In another embodiment of the present disclosure, the at least one phytocannabinoid consists of (or “is”) cannabidivarin, cannabinol, cannabigerol, cannabichromene or combinations thereof.

Of the individual phytocannabinoids administered to MM cell lines, the efficacy in reducing cell viability was generally highest for CBDV. Accordingly, in an embodiment, the at least one phytocannabinoid consists essentially of or consists of (or “is”) cannabidivarin. In another embodiment, the at least one phytocannabinoid consists essentially of cannabidivarin. In a further embodiment, the at least one phytocannabinoid consists of (or “is”) cannabidivarin.

However, a dose dependent effect in all MM cell lines was also observed for the other individual phytocannabinoids CBN, CBG and CBC tested in the present examples. Accordingly, in an embodiment, the at least one phytocannabinoid consists essentially of or consists of (or “is”) cannabinol. In another embodiment, the at least one phytocannabinoid consists essentially of cannabinol. In a further embodiment, the at least one phytocannabinoid consists of (or “is”) cannabinol. In an embodiment, the at least one phytocannabinoid consists essentially of or consists of (or “is”) cannabigerol. In another embodiment, the at least one phytocannabinoid consists essentially of cannabigerol. In a further embodiment, the at least one phytocannabinoid consists of (or “is”) cannabigerol. In an embodiment, the at least one phytocannabinoid consists essentially of or consists of (or “is”) cannabichromene. In another embodiment, the at least one phytocannabinoid consists essentially of cannabichromene. In a further embodiment, the at least one phytocannabinoid consists of (or “is”) cannabichromene.

The combination of CBG with CBC was surprisingly observed to generally have the highest efficacy in reducing cell viability for MM cell lines followed by combinations comprising CBN and one of the other phytocannabinoids. Several of the particular doses for these combinations were observed to be more effective compared to the sum of the individual compounds.

Accordingly, in an embodiment, the at least one phytocannabinoid consists essentially of or consists of (or “is”) a combination of cannabigerol and cannabichromene. In another embodiment, the at least one phytocannabinoid consists essentially of a combination of cannabigerol and cannabichromene. In a further embodiment, the at least one phytocannabinoid consists of (or “is”) a combination of cannabigerol and cannabichromene.

In an embodiment, the combination of at least one phytocannabinoid that is cannabidivarin, cannabinol, cannabigerol or cannabichromene is a combination comprising cannabinol. In another embodiment, the combination further consists of cannabigerol. For example, the at least one phytocannabinoid consists essentially of or consists of (or “is”) a combination of cannabinol and cannabigerol. In another embodiment, the at least one phytocannabinoid consists essentially of a combination of cannabinol and cannabigerol. In a further embodiment, the at least one phytocannabinoid consists of (or “is”) a combination of cannabinol and cannabigerol. Alternatively, in a further embodiment, the combination further consists of cannabichromene. For example, the at least one phytocannabinoid consists essentially of or consists of (or “is”) a combination of cannabinol and cannabichromene. In another embodiment, the at least one phytocannabinoid consists essentially of a combination of cannabinol and cannabichromene. In a further embodiment, the at least one phytocannabinoid consists of (or “is”) a combination of cannabinol and cannabichromene. Alternatively, in another embodiment, the combination further consists of cannabidivarin. For example, the at least one phytocannabinoid consists essentially of or consists of (or “is”) a combination of cannabinol and cannabidivarin. In another embodiment, the at least one phytocannabinoid consists essentially of a combination of cannabinol and cannabidivarin. In a further embodiment, the at least one phytocannabinoid consists of (or “is”) a combination of cannabinol and cannabidivarin.

In an embodiment, the combination of at least one phytocannabinoid that is cannabidivarin, cannabinol, cannabigerol or cannabichromene is a combination comprising cannabigerol. In another embodiment of the present disclosure, the combination of at least one phytocannabinoid that is cannabidivarin, cannabinol, cannabigerol or cannabichromene is a combination comprising cannabichromene. In a further embodiment, the combination of at least one phytocannabinoid that is cannabidivarin, cannabinol, cannabigerol or cannabichromene is a combination comprising cannabidivarin.

In an embodiment, the at least one phytocannabinoid consists essentially of or consists of (or “is”) a combination of cannabigerol, cannabinol and cannabidivarin. In another embodiment, the at least one phytocannabinoid consists essentially of a combination of cannabigerol, cannabinol and cannabidivarin. In a further embodiment, the at least one phytocannabinoid consists of (or “is”) a combination of cannabigerol, cannabinol and cannabidivarin. In another embodiment, the at least one phytocannabinoid consists essentially of or consists of (or “is”) a combination of cannabichromene, cannabinol and cannabidivarin. In another embodiment, the at least one phytocannabinoid consists essentially of a combination of cannabichromene, cannabinol and cannabidivarin. In a further embodiment, the at least one phytocannabinoid consists of (or “is”) a combination of cannabichromene, cannabinol and cannabidivarin. In another embodiment, the at least one phytocannabinoid consists essentially of or consists of (or “is”) a combination of cannabichromene, cannabinol and cannabigerol. In another embodiment, the at least one phytocannabinoid consists essentially of a combination of cannabichromene, cannabinol and cannabigerol. In a further embodiment, the at least one phytocannabinoid consists of (or “is”) a combination of cannabichromene, cannabinol and cannabigerol. In another embodiment, the at least one phytocannabinoid consists essentially of or consists of (or “is”) a combination of cannabichromene, cannabidivarin and cannabigerol. In another embodiment, the at least one phytocannabinoid consists essentially of a combination of cannabichromene, cannabidivarin and cannabigerol. In a further embodiment, the at least one phytocannabinoid consists of (or “is”) a combination of cannabichromene, cannabidivarin and cannabigerol.

In an embodiment, the at least one phytocannabinoid consists essentially of or consists of (or “is”) a combination of cannabigerol, cannabinol, cannabidivarin and cannabichromene. In another embodiment, the at least one phytocannabinoid consists essentially of a combination of cannabigerol, cannabinol, cannabidivarin and cannabichromene. In a further embodiment of the present disclosure, the at least one phytocannabinoid consists of (or “is”) a combination of cannabigerol, cannabinol, cannabidivarin and cannabichromene.

In an embodiment, the at least one phytocannabinoid is an individual phytocannabinoid. In another embodiment, the at least one phytocannabinoid is a combination of two phytocannabinoids. In a further embodiment, the at least one phytocannabinoid is a combination of three phytocannabinoids. In another embodiment of the present disclosure, the at least one phytocannabinoid is a combination of four phytocannabinoids.

In an embodiment, the plasma cell neoplasm is benign (i.e. not cancerous). In another embodiment, the plasma cell neoplasm is malignant (i.e. is a cancer).

In an embodiment, the plasma cell neoplasm is multiple myeloma.

In an embodiment, the subject has myeloma bone disease and the at least one phytocannabinoid is further for treatment of the myeloma bone disease.

Accordingly, the present disclosure also includes a method of treating myeloma bone disease in a subject in need thereof, comprising administering an effective amount of at least one phytocannabinoid to the subject, with the proviso that the at least one phytocannabinoid does not comprise cannabidiol or tetrahydrocannabinol. The present disclosure also includes a method of treating myeloma bone disease in a subject in need thereof, comprising administering an effective amount of at least one phytocannabinoid to the subject, with the proviso that the at least one phytocannabinoid does not comprise cannabidiol. The present disclosure also includes a method of treating myeloma bone disease in a subject in need thereof, comprising administering an effective amount of at least one phytocannabinoid to the subject, with the proviso that the at least one phytocannabinoid does not comprise tetrahydrocannabinol.

The present disclosure also includes a use of an effective amount of at least one phytocannabinoid for treatment of myeloma bone disease in a subject in need thereof, with the proviso that the at least one phytocannabinoid does not comprise cannabidiol or tetrahydrocannabinol. The present disclosure also includes a use of an effective amount of at least one phytocannabinoid for treatment of myeloma bone disease in a subject in need thereof, with the proviso that the at least one phytocannabinoid does not comprise cannabidiol. The present disclosure also includes a use of an effective amount of at least one phytocannabinoid for treatment of myeloma bone disease in a subject in need thereof, with the proviso that the at least one phytocannabinoid does not comprise tetrahydrocannabinol.

The present disclosure also includes a use of an effective amount of at least one phytocannabinoid for preparation of a medicament for treatment of myeloma bone disease in a subject in need thereof, with the proviso that the at least one phytocannabinoid does not comprise cannabidiol or tetrahydrocannabinol. The present disclosure also includes a use of an effective amount of at least one phytocannabinoid for preparation of a medicament for treatment of myeloma bone disease in a subject in need thereof, with the proviso that the at least one phytocannabinoid does not comprise cannabidiol. The present disclosure also includes a use of an effective amount of at least one phytocannabinoid for preparation of a medicament for treatment of myeloma bone disease in a subject in need thereof, with the proviso that the at least one phytocannabinoid does not comprise tetrahydrocannabinol.

The present disclosure also includes at least one phytocannabinoid for use to treat myeloma bone disease in a subject in need thereof, with the proviso that the at least one phytocannabinoid does not comprise cannabidiol or tetrahydrocannabinol. The present disclosure also includes at least one phytocannabinoid for use to treat myeloma bone disease in a subject in need thereof, with the proviso that the at least one phytocannabinoid does not comprise cannabidiol. The present disclosure also includes at least one phytocannabinoid for use to treat myeloma bone disease in a subject in need thereof, with the proviso that the at least one phytocannabinoid does not comprise tetrahydrocannabinol.

The at least one phytocannabinoid can be any suitable phytocannabinoid or combination thereof, for example, as described in the embodiments of the present disclosure in respect to the treatment of plasma cell neoplasms. For example, in an embodiment, the at least one phytocannabinoid consists essentially of or consists of (or “is”) cannabinol. In another embodiment, the at least one phytocannabinoid consists essentially of cannabinol. In a further embodiment, the at least one phytocannabinoid consists of (or “is”) cannabinol. In another embodiment, the at least one phytocannabinoid consists essentially of or consists of (or “is”) cannabigerol. In another embodiment, the at least one phytocannabinoid consists essentially of cannabigerol. In a further embodiment, the at least one phytocannabinoid consists of (or “is”) cannabigerol. In an embodiment, the at least one phytocannabinoid consists essentially of or consists of (or “is”) a combination of cannabigerol and cannabinol. In another embodiment, the at least one phytocannabinoid consists essentially of a combination of cannabigerol and cannabinol. In a further embodiment, the at least one phytocannabinoid consists of (or “is”) a combination of cannabigerol and cannabinol.

In an embodiment, the at least one phytocannabinoid is administered or for use in combination with at least one other anticancer treatment. The other anticancer treatment can be any other anticancer treatment suitable for treatment of plasma cell neoplasms such as multiple myeloma. In an embodiment, the other anticancer treatment is a proteasome inhibitor (e.g. bortezomib, carfilzomib or combinations thereof), an immunomodulator (e.g. thalidomide, lenalidomide or combinations thereof) or combinations thereof. In an embodiment, the at least one other anticancer treatment comprises administration to the subject or use of a proteasome inhibitor. In an embodiment, the proteasome inhibitor is bortezomib. In another embodiment, the proteasome inhibitor is carfilzomib. In an embodiment, the at least one other anticancer treatment comprises administration to the subject or use of an immunomodulator. In an embodiment, the immunomodulator is thalidomide. In another embodiment, the immunomodulator is lenalidomide.

In an embodiment, the subject is a human.

The at least one phytocannabinoid is administered to a subject or used in a suitable form depending on the selected route of administration or use, as will be understood by those skilled in the art. In an embodiment, the at least one phytocannabinoid is administered to the subject or for use in the form of a suitable composition (e.g. a pharmaceutical composition) comprising the at least one phytocannabinoid and a suitable carrier (e.g. a pharmaceutically acceptable carrier). In another embodiment, the at least one phytocannabinoid is administered to the subject or used by enteral or parenteral routes, and the at least one phytocannabinoid formulated accordingly. Conventional procedures and ingredients for the selection and preparation of suitable compositions are described, for example, in Remington: The Science and Practice of Pharmacy (2020—23rd edition) and in The United States Pharmacopeia: The National Formulary (USP 43 NF 37) published in 2019. Enteral administration or use includes all suitable routes involving the gastrointestinal tract, for example, oral, buccal, sublingual, nasal and rectal. In an embodiment of the present disclosure, the enteral administration or use of the at least one phytocannabinoid is oral administration or use; i.e. the at least one phytocannabinoid is administered orally or is for oral use, as the as the case may be. Formulations suitable for oral administration or use may be prepared by methods known to a person skilled in the art. For example, in an embodiment, the at least one phytocannabinoid is administered to the subject or for use in the form of a suitable composition (e.g. a pharmaceutical composition) comprising the at least one phytocannabinoid and a suitable oil (e.g. a pharmaceutically acceptable oil). Suitable oils include but are not limited to sunflower oil, hemp seed oil, olive oil and/or mixtures thereof. In another embodiment, the at least one phytocannabinoid is administered to the subject or for use in the form of a gastro-resistant capsule comprising the at least one phytocannabinoid. Parenteral administration or use includes intravesical, intravenous, intraperitoneal, subcutaneous, intramuscular, transepithelial, intrapulmonary, intrathecal, and topical modes of administration or use. Formulations suitable for parenteral administration or use may be prepared by known methods by a person skilled in the art. In an embodiment, the at least one phytocannabinoid is administered to the subject or for use intrapulmonarily via inhalation (e.g. inhalation via vaporization). For example, in an embodiment, the at least one phytocannabinoid is administered to the subject or for use in a form suitable for inhalation via vaporization such as in a suitable composition (e.g. a pharmaceutical composition) comprising the at least one phytocannabinoid and a suitable vehicle such as but not limited to propylene glycol. Forms suitable for inhalation via vaporization can be administered to the subject or used with a suitable vaporizer device.

Treatment methods comprise administering to a subject or use of an effective amount of the at least one phytocannabinoid and optionally consist of a single administration or use, or alternatively comprise a series of administrations or uses. For example, the at least one phytocannabinoid is administered or used at least once a week. However, in another embodiment, the at least one phytocannabinoid is administered to the subject or used from one time per three weeks or one time per week to once daily for a given treatment or use. In another embodiment, the at least one phytocannabinoid is administered to the subject or used once daily. In another embodiment, the at least one phytocannabinoid is administered or used 2, 3, 4, 5 or 6 times daily. The length of the treatment period depends on a variety of factors, such as the severity of the disease, disorder or condition such as multiple myeloma (e.g. disease stage), the age and/or sex of the subject, and the activity and/or formulation of the at least one phytocannabinoid and/or a combination thereof. It will also be appreciated that the effective amount of the at least one phytocannabinoid used for the treatment or use may increase or decrease over the course of a particular treatment regime or use. Changes in dosage may result and become apparent by standard diagnostic assays known in the art. In some embodiments of the present disclosure, chronic administration or use may be required. For example, the at least one phytocannabinoid is administered or used in an amount and for a duration sufficient to treat the subject.

In embodiments, wherein the at least one phytocannabinoid is a combination of at least two phytocannabinoids, the phytocannabinoids are either used or administered separately in time and/or in mode of administration or use (i.e. different routes of administration or use) or they are administered or for use together in the same pharmaceutical preparation and/or at the same time, which may depend, for example, on the identity of the phytocannabinoids.

In an embodiment, the at least two phytocannabinoids are used or administered separately in time and/or in mode of administration or use.

In another embodiment, the at least two phytocannabinoids are administered or for use contemporaneously. As used herein, contemporaneous administration or use, for example, of two substances to a subject means providing the first phytocannabinoid and the second phytocannabinoid so that the pharmacological effects of the first phytocannabinoid and the second phytocannabinoid are present in the subject at the same time. The exact details of the administration or use will depend on the pharmacokinetics of the first phytocannabinoid and the second phytocannabinoid in the presence of each other, and can include administering or use of the first phytocannabinoid and the second phytocannabinoid within a few hours of each other, or even administering or use of the first phytocannabinoid and the second phytocannabinoid within 24 hours, or 48 hours or greater of administration or use of the other, if the pharmacokinetics are suitable. Design of suitable dosing regimens is routine for one skilled in the art. In some embodiments, the at least two phytocannabinoids are administered or used substantially simultaneously; i.e. within minutes of each other or in a single composition that comprises both substances. In another embodiment, the at least two phytocannabinoids are administered to a subject or for use in a non-contemporaneous fashion. In a further embodiment, the at least two phytocannabinoids are administered to a subject or for use in a contemporaneous fashion followed by, or alternating with, administration or use in a non-contemporaneous fashion.

Similarly, in embodiments, wherein the at least one phytocannabinoid is administered or for use in combination with at least one other anticancer treatment, the phytocannabinoid(s) and other anticancer treatment(s) are either used or administered separately in time and/or in mode of administration or use (i.e. different routes of administration or use) or they are administered or for use together in the same pharmaceutical preparation and/or at the same time, which may depend, for example, on the identity of the phytocannabinoid(s) and/or other anticancer treatment(s).

The dosage of the at least one phytocannabinoid can vary depending on many factors such as the pharmacodynamic properties of the phytocannabinoid or combination thereof, the mode of administration or use, the age, health and weight of the subject, the nature and extent of the symptoms of the disease, disorder or condition such as multiple myeloma, the frequency of the treatment or use and the type of concurrent treatment or use, if any, and/or the clearance rate of the phytocannabinoid in the subject. One of skill in the art can determine the appropriate dosage having regard to the above factors. In an embodiment, the at least one phytocannabinoid is administered or used initially in a suitable dosage that is optionally adjusted as desired, depending on the clinical response. As a representative example, oral dosages of the at least one phytocannabinoid may range from less than 1 mg per day to 1000 mg per day for a human subject. In an embodiment of the present disclosure, the at least one phytocannabinoid is formulated in a pharmaceutical composition suitable for oral administration or use and the compounds are, for example, present in an amount of about 0.001, 0.01, 0.1, 0.25, 0.5, 0.75, 1.0, 5.0, 7.5, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per dose. In another embodiment of the present disclosure, the at least one phytocannabinoid is administered or used in a single daily dose or the total daily dose may be divided into two, three or four daily doses.

The following non-limiting examples are illustrative of the present disclosure:

### EXAMPLES

### Example 1: Effect of Phytocannabinoids and Combinations Thereof on MM Cell Lines

This study evaluated the effects of four cannabinoids named: Cannabigerol (CBG), Cannabinol (CBN), Cannabichromene (CBC), Cannabidivarin (CBDV) alone and in combination, in regulating cell survival in human multiple myeloma (MM) cell lines.

I. Materials and Methods

MTT cytotoxicity assay: MM cell lines (4×10⁴ cells/ml) were seeded in 96-well plates, in a final volume of 100 μl/well. After 24 hours, CBG, CBC, CBN, CBDV alone and in combination or vehicle were added at different concentrations, for 72 hours. At least six replicates were used for each treatment. At the indicated time point, cell viability was assessed by adding 0.8 mg/ml of 3-[4,5-dimethylthiazol-2-yl]-2,5 diphenyl tetrazolium bromide (MTT) (Sigma-Aldrich) to the media. After 3 hours, the plates were centrifuged, the supernatant was removed, and the pellet was solubilized with 100 μl/well dimethyl sulfoxide (DMSO). The absorbance of the samples against a background control (medium alone) was measured at 570 nm using an ELISA reader microliter plate (BioTek™ Instruments, Winooski, Vt., USA).

RNA isolation, reverse transcription and quantitative real-time PCR: Total RNA from myeloma cells was extracted with RNeasy Mini Kit (Qiagen) and cDNA was synthesized using the High-Capacity cDNA Archive Kit (Applied Biosystems™, Foster City, Pa.) according to the instructions. Quantitative real-time polymerase chain reactions (qRT-PCR) were performed with QuantiTect™ Primer Assays for Human Cannabinoid receptor 1 (CNR1, CB1), Human Cannabinoid receptor 2 (CNR2, CB2) and transient receptor potential (TRP) channel profile was performed using the TaqMan™ array 96 (Applied Biosystems) according to the manufacturer's protocol. Measurement of glyceraldehyde 3-phosphate dehydrogenase (GAPDH) levels were used to normalize mRNA contents and target gene levels were calculated by the 2^{-ΔΔct} method.

Western blot for cannabinoid ligands: Lysates from myeloma cell lines were obtained with lysis buffer [composed of tris(hydroxymethyl)aminomethane (TRIS) 1M pH 7.4, NaCl 1M, ethylene glycol-bis(β-aminoethyl ether)-N,N,N′,N′-tetraacetic acid (EGTA) 10 mM, NaF 100 mM, deoxycholate 2%, ethylenediaminetetraacetic acid (EDTA) 100 mM, Triton™ X-100 10%, glycerol, sodium dodecyl sulfate (SDS) 10%, Na₂P₂O₇ 1M, Na₃VO₄ 100 mM, phenylmethylsulfonyl fluoride (PMSF) 100 mM, cocktail of enzyme inhibitors and H₂O]. Lysates were separated on a SDS polyacrylamide gel, transferred onto Hybond™-C extra membranes (GE Healthcare), blocked with 5% low-fat dry milk in phosphate-buffered saline 0.1% Tween™ 20 overnight at 4° C., immunoblotted with mouse anti-CB1 (1:500, Santa Cruz), rabbit anti-CB2 (1:200, Cayman Chemical), mouse anti-TRPV1 (1:200, Santa Cruz), mouse anti-TRPV2 (1:200, Santa Cruz), rabbit anti-TRPV3 (0.5 μg/ml, Boster Biological technology), rabbit anti-TRPV4 (1:500, Assay Biotechnology Company), goat anti-TRPA1 (1:300, Santa Cruz), rabbit anti-TRPM8 (0.5 μg/ml, Boster Biological technology) and mouse anti-glyceraldehyde-3-phosphate dehydrogenase (GAPDH, 1:3000, OriGene, Rockville, Md., USA) antibodies overnight or 1 h according to manufacturers' protocols and then incubated with their respective horseradish peroxidase (HRP)-conjugated anti-rabbit, anti-mouse (1:2000, Cell Signaling) or anti-goat (1:1000, Santa Cruz) antibodies for 1 hour. Peroxidase activity was visualized with the LiteAblot™ PLUS or LiteAblot™ TURBO (EuroClone, Milan, Italy) kits and densitometric analysis was carried out by a Chemidoc using the Quantity One software (Bio-Rad).

Western blot analysis for H2AX: 4×10⁴ cells were plated in 6-well plates and treated daily for 48 hours with CBG, CBN or CBDV (25 μM) or CBC (75 μM). Ten micrograms of the myeloma cell line lysates were then separated on a SDS-polyacrylamide gel, transferred onto Hybond-C extra membranes (GE Healthcare), blocked with 5% of Bovine Serum Albumin (BSA) in phosphate buffered saline (PBS)-Tween 20, immunoblotted with rabbit anti-phospho-histone H2AX (Ser139) (1:1000, #9718, Cell Signaling) and rabbit anti-glyceraldehyde-3-phosphate dehydrogenase (GAPDH, 1:3000, #2118, OriGene, Rockville, Md., USA) antibodies incubated overnight and then incubated with their respective HRP-conjugated anti-rabbit (1:2000, Cell Signaling) antibodies for 1 hour. The detection was performed using the LiteAblot™ PLUS or the LiteAblot™ TURBO (EuroClone, Milano, Italy) kits and densitometric analysis was carried out by evaluating three independent experiments by a Chemidoc using the Quantity One software (BioRad, Hercules, USA). H2AX densitometry values were normalized to GAPDH used as loading control and normalized with non-treated cells. Densitometric values shown are the mean±SE of three separate experiments.

Apoptosis assays and PI staining: Cell death was evaluated using the Annexin V-FITC Apoptosis detection Kit (eBioscience) followed by biparametric fluorescence-activated cell sorting (FACS) analysis. Cells, at a density of 4×10⁴ cells/ml, were treated with CBG, CBC, CBN or CBDV for a maximum of 48 hours, with daily administration, and then incubated with Annexin V-FITC and propidium iodide (PI), following the protocol provided. The percentage of positive cells determined over 10,000 events was analysed on a FACScan cytofluorimeter using the CellQuest software.

Alkaline Comet Assay: U266, SKO and RPMI myeloma cell lines were plated in 6-well plates (4×10⁴ cell/well) one day before treatment exposure. Semi-confluent cultures were exposed for 48 hrs with 25 μM of CBG, CBN or CBDV or 75 μM of CBC. Controls, treated with vehicle, were included in all series. The Comet assay was performed under alkaline conditions following the ABCAM™ protocol. Briefly, after exposure to the phytocannabinoid, the cells were incubated for 48 hrs in Roswell Park Memorial Institute (RPMI) medium. Then the cells were resuspended in 1×PBS and added to 75 μL of molten (37° C.) 0.5% low-melting-point agarose gel to achieve a cell concentration of 1×10⁵ cells/mL. The agarose was pipetted onto the Comet slides. Slides were stored in the dark at 4° C. for 10 min before adding pre-chilled lysis buffer for 45 min. The slides were immersed in freshly prepared alkaline solution (0.25 M NaOH containing 0.1 μM EDTA, pH 12.6) for 30 min at room temperature. Slides were then removed and washed twice with alkaline solution for 5 min. Gel electrophoresis was performed at 1 V/cm for 30 min (running amperage 3-5 mA with the distance between the two electrodes of 25 cm). The Comet slides were washed with 70% ethanol for 5 min and air-dried for one hour at room temperature. 100 μL of diluted SYBR™ Green solution was placed onto each dried agarose circle. The slides were then read with a fluorescence microscope (LEIKA).

II. Results

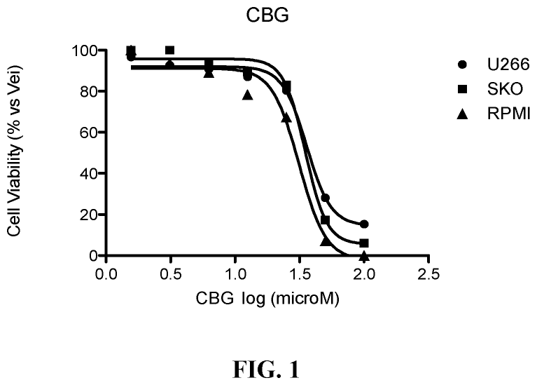

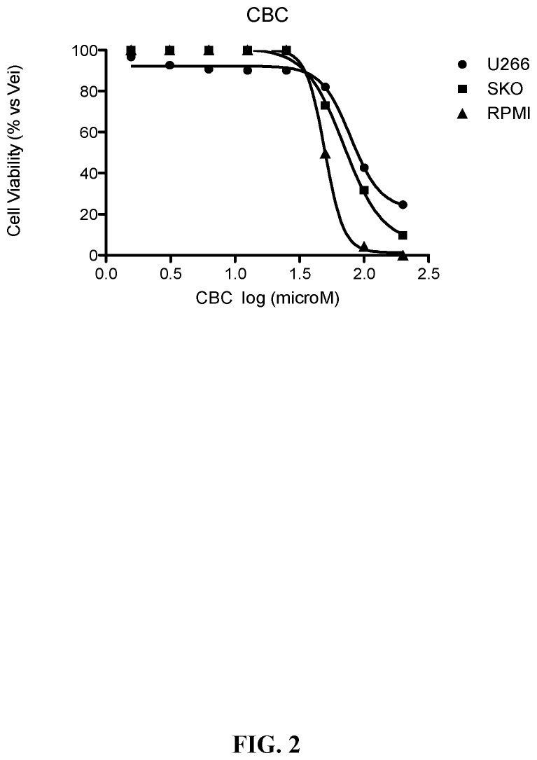

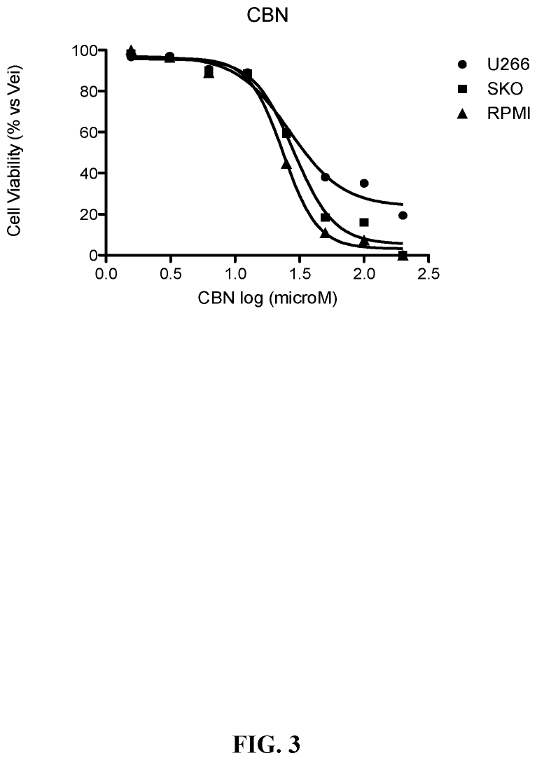

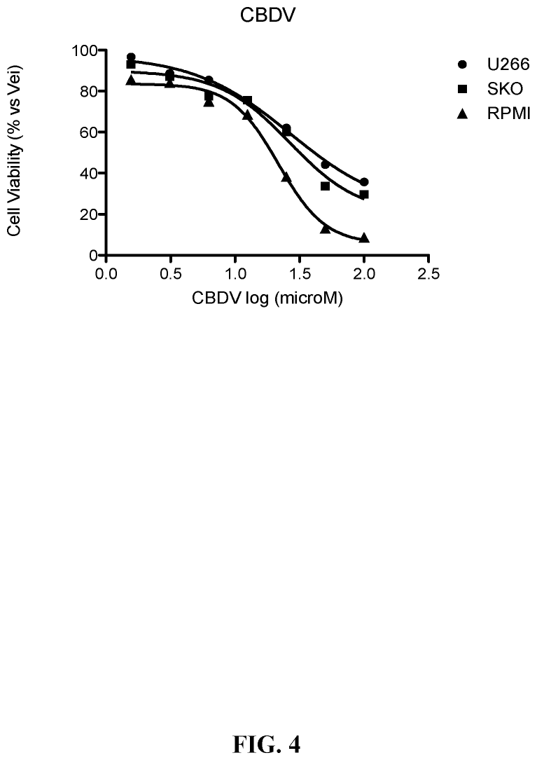

Phytocannabinoids induce cytotoxicity in MM cell lines: The effect of CBG, CBC, CBN and CBDV in reducing cell viability was evaluated at 72 hours, post daily administration, in MM cell lines. Cells were treated with different doses of CBG, CBC, CBN and CBDV (up to 100 μM) and percentage of cell viability was evaluated by the MTT assay. The results showed a dose dependent effect in all MM cell lines for all of the phytocannabinoids (FIGS. 1-4, Table 1).

TABLE 1
IC₅₀ (μM) of individual phytocannabinoids in MM cell lines.

IC₅₀ (μM) ± S.D.

| Phytocannabinoid | U266 | SKO | RPMI |
|---|---|---|---|
| CBG | 35.91 ± 2.2 | 35.02 ± 1.8 | 31.36 ± 1.9 |
| CBC | 78.8 ± 3.4 | 69.7 ± 3.5 | 49.6 ± 2.1 |
| CBN | 26.51 ± 1.8 | 28.5 ± 2.2 | 23.64 ± 0.9 |
| CBDV | 26.71 ± 1.3 | 26.52 ± 2.1 | 21.68 ± 1.1 |

IC₅₀ values, relative to daily administration, were reported. The efficacy in reducing cell viability was generally highest for CBDV, followed by CBN, CBG and CBC.

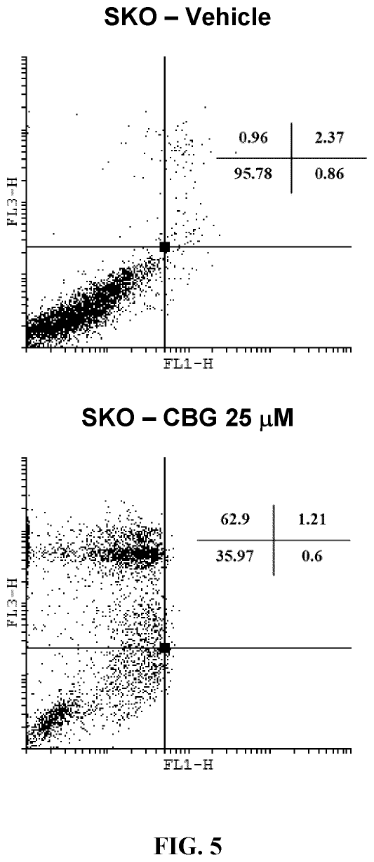

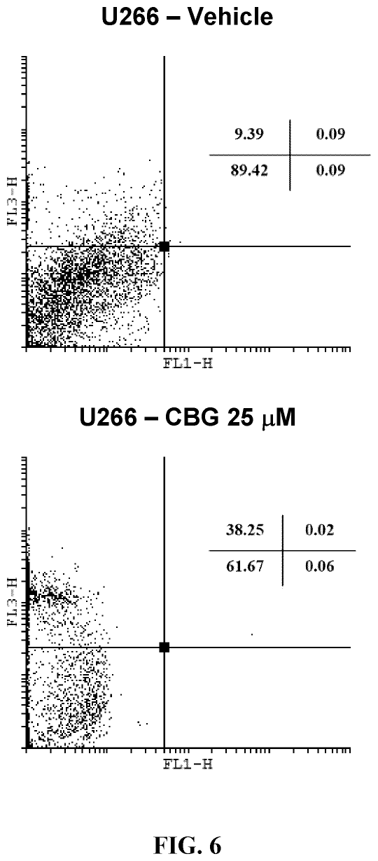

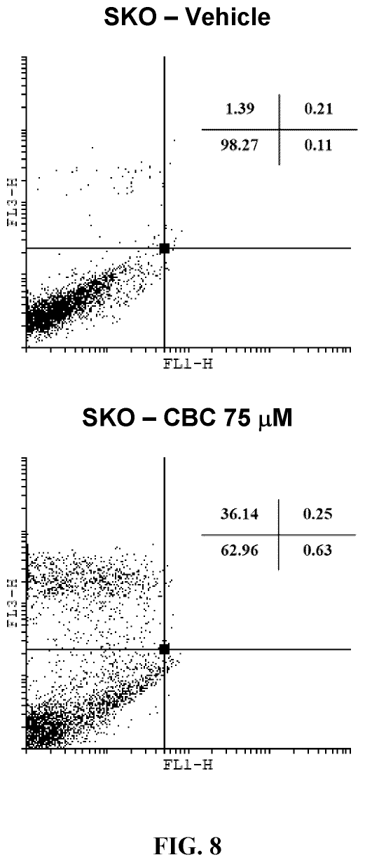

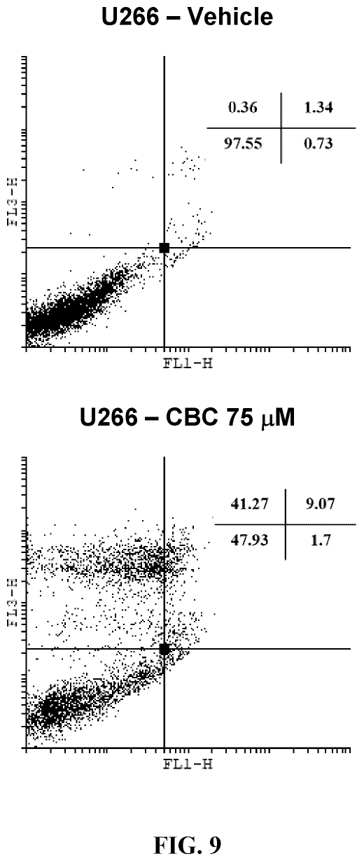

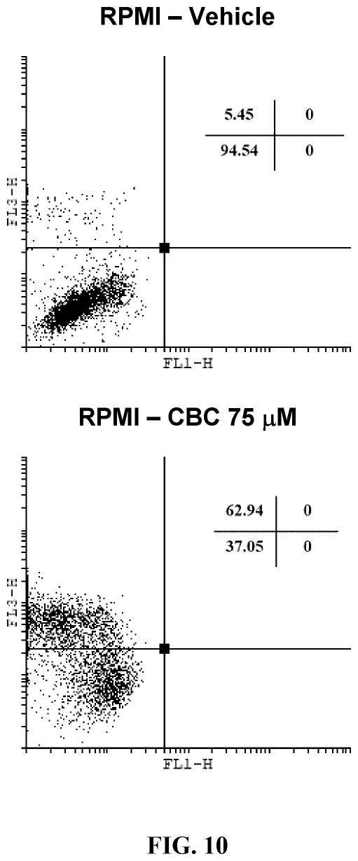

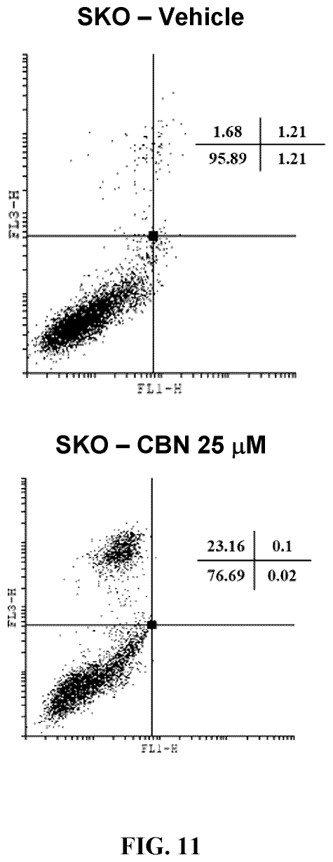

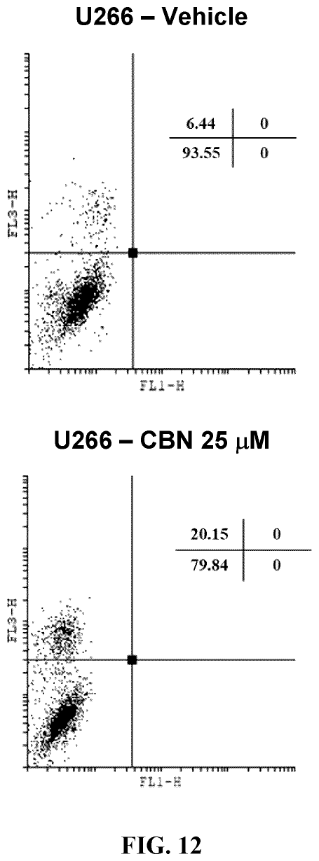

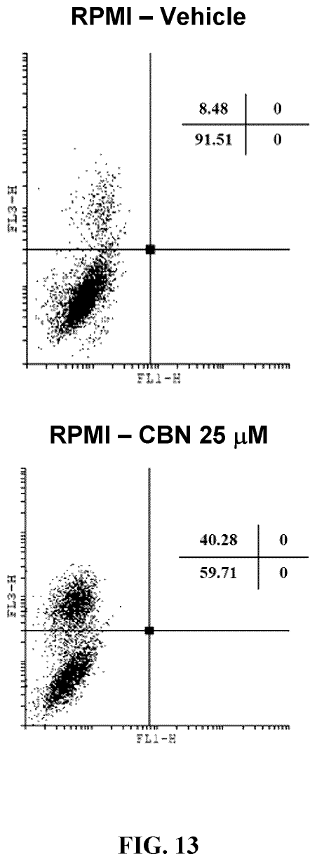

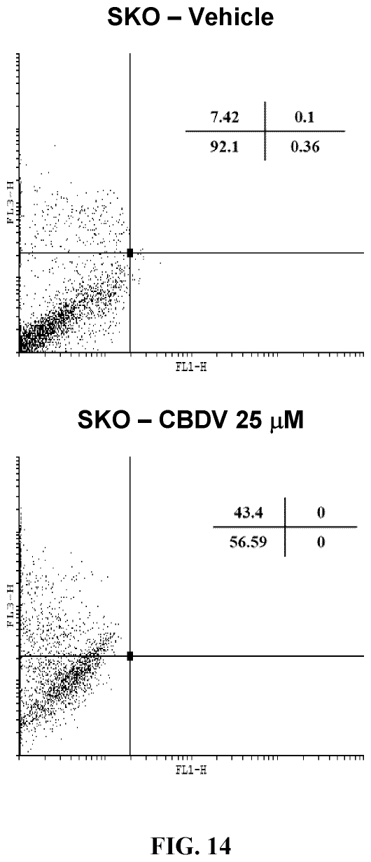

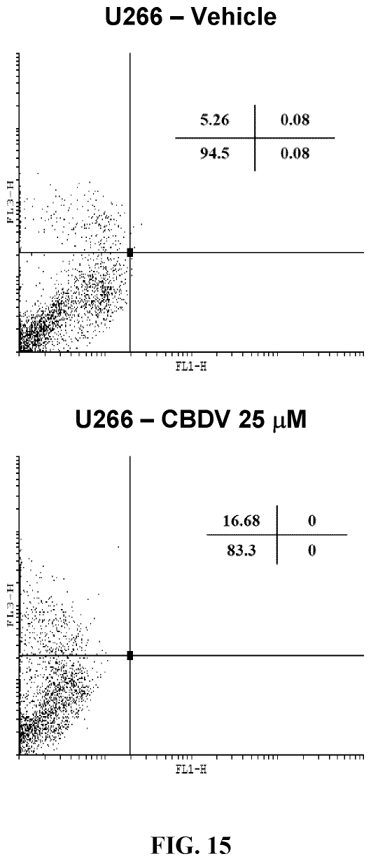

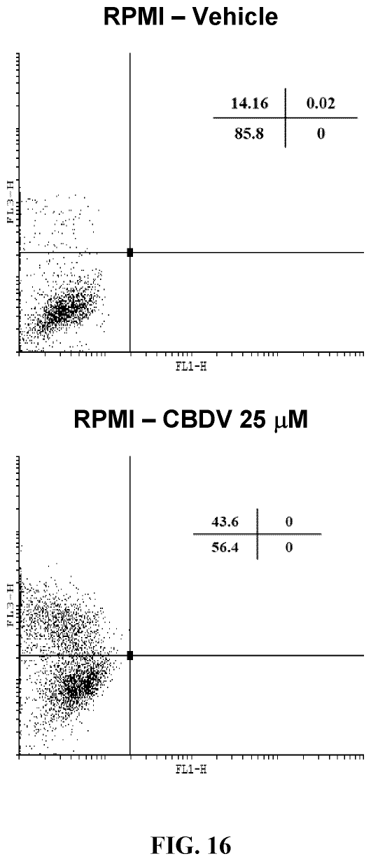

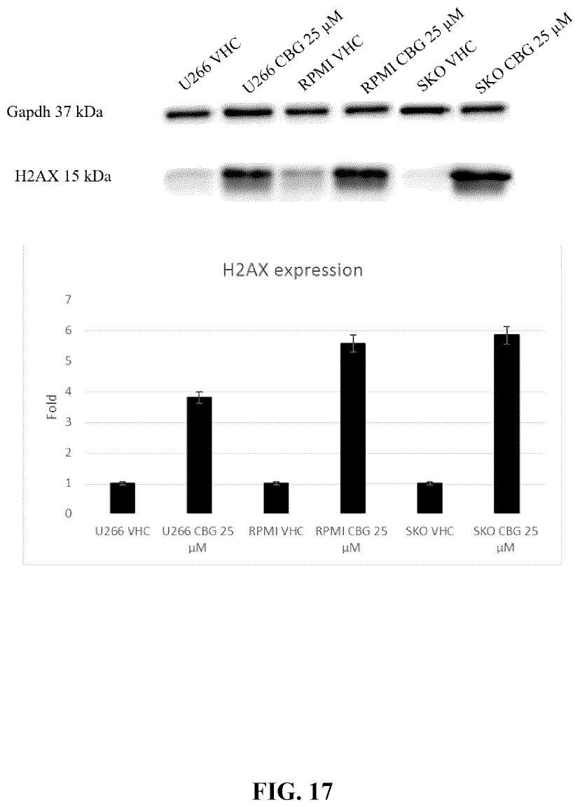

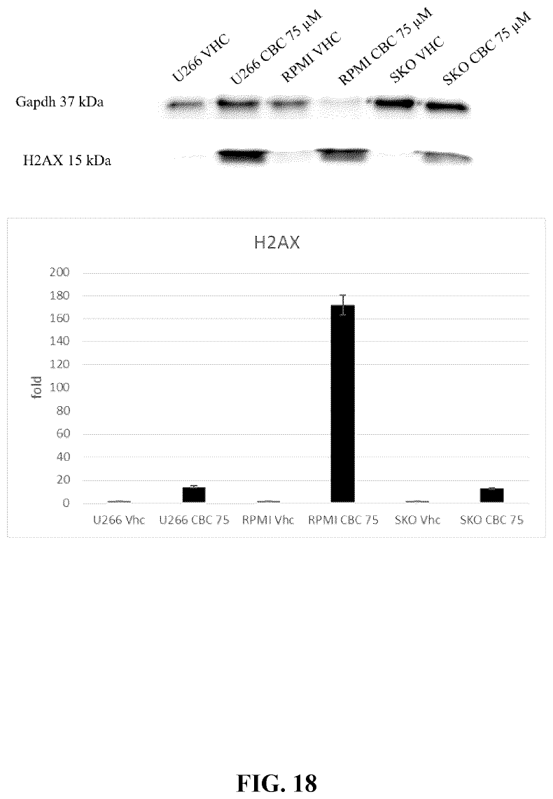

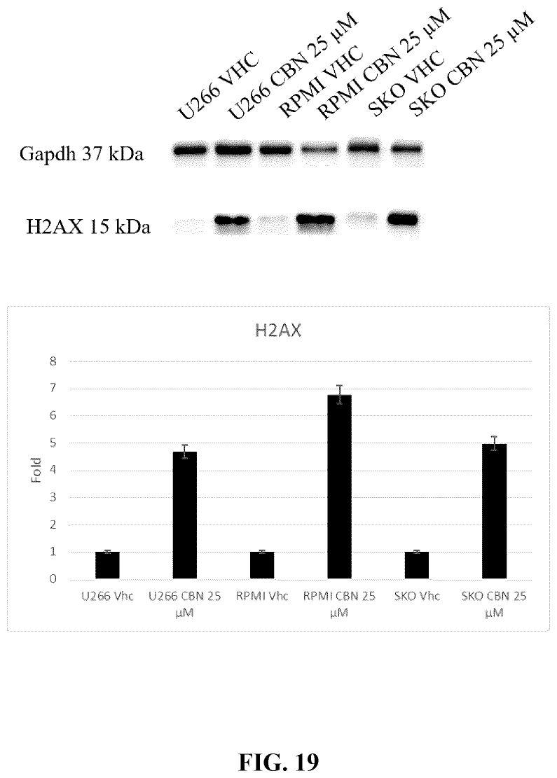

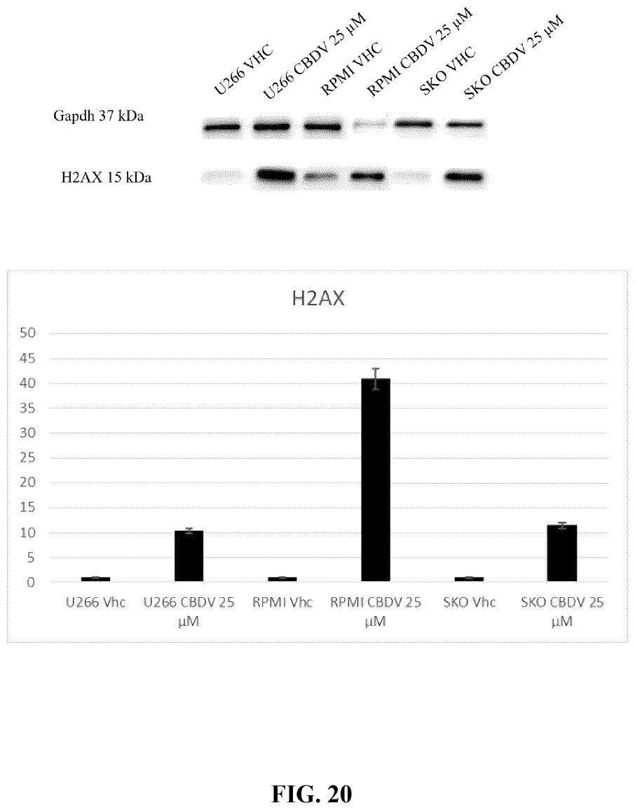

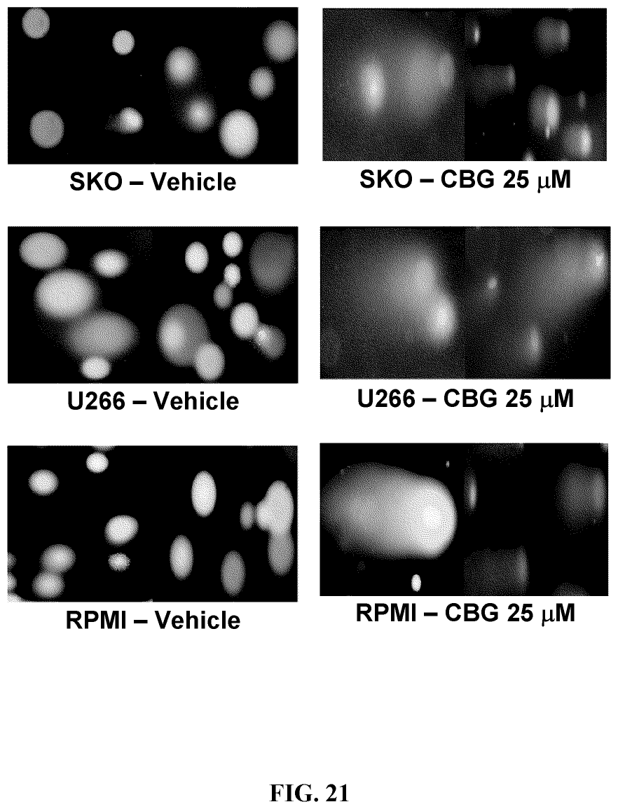

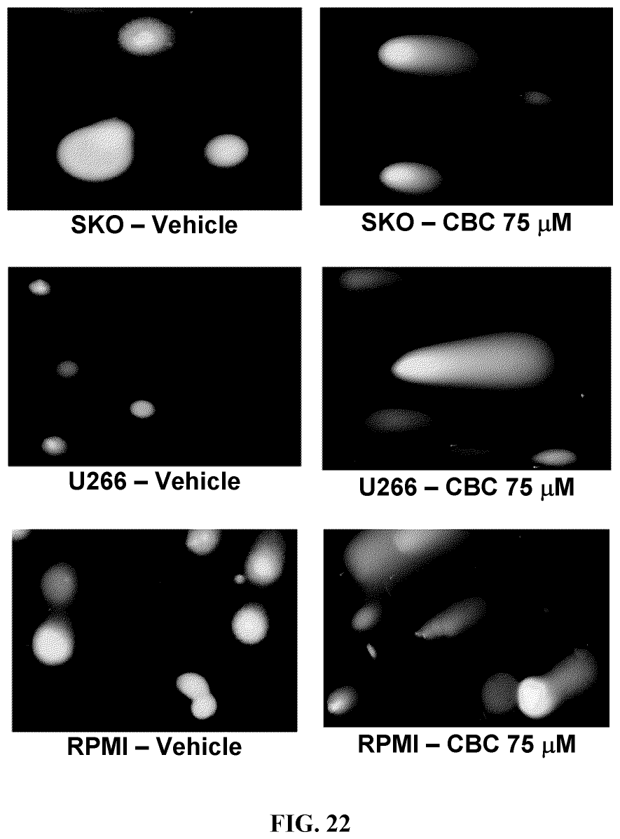

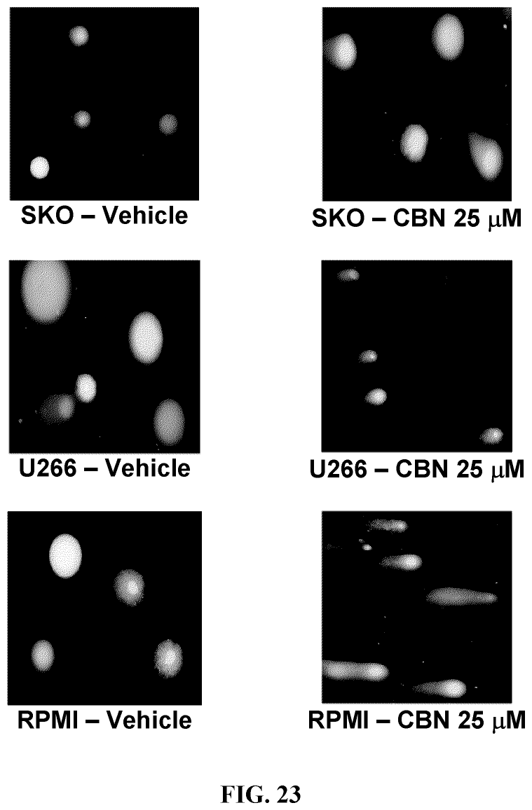

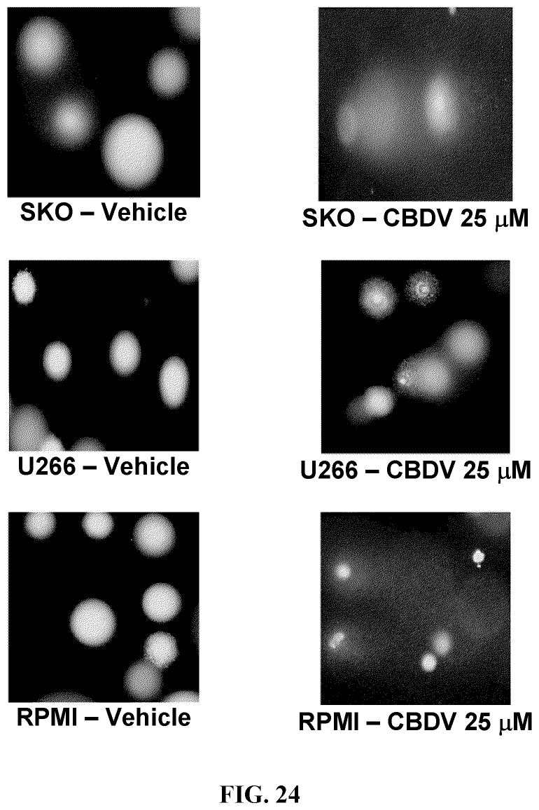

Phytocannabinoids induce necrotic cell death in MM cell lines: To evaluate the cytotoxic mechanism induced by the phytocannabinoids, MM cell lines were treated daily with a cytotoxic dose for each phytocannabinoid (CBG: 25 μM; CBC 75 μM; CBN 25 μM; CBDV 25 μM) for 48 hours and then Annexin-V/PI cell positivity was evaluated by FACS analysis. The results evidenced that all of the phytocannabinoids induced necrotic cell death but had different efficacies (FIGS. 5-16). To confirm the necrotic cell death mechanism, MM cell lines were treated as above and DNA damage was evaluated by H2AX (a marker to examine the DNA damage produced and the subsequent repair of the DNA lesion) Western blot analysis and Comet assay (permits the analysis of DNA damage in single cell preparations). The result confirmed the DNA damage induced by the phytocannabinoid analysed. In particular, an increase of H2AX levels were observed by Western blot (FIGS. 17-20) and DNA damage by Comet assay that evidenced structures resembling comets in the treated cell lines (FIGS. 21-24).

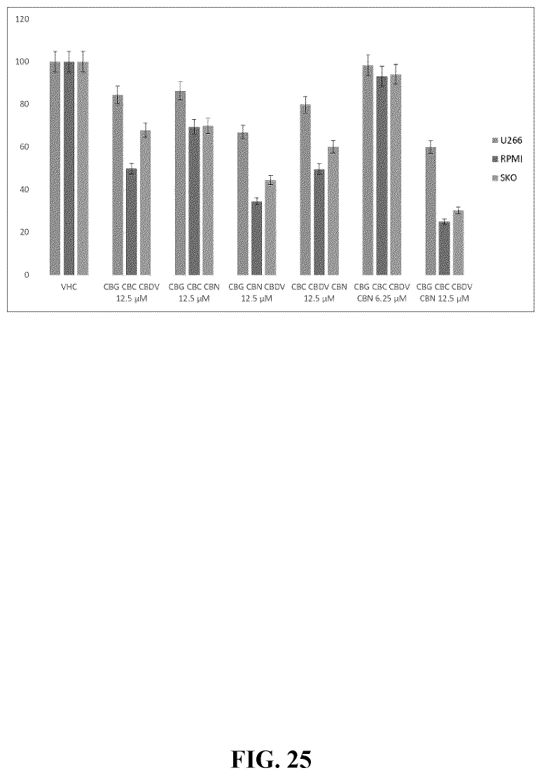

Phytocannabinoid combinations reduce cell viability in MM cell lines: To evaluate a potential synergism between the phytocannabinoids tested, a MTT assay combining CBG, CBC, CBN and CBDV at lower cytotoxic doses (see Tables 1-2) in pairs (Table 2), triplets (Table 3) and all four together (Table 4) was performed. The cannabinoids were administered daily for 72 hours, as in previous experiments at the desired doses. Each combination was evaluated in six wells and in two separate experiments. MTT assay was used to analyze the cell cytotoxicity. The values in the tables are represented as % of cell viability compared to vehicle-treated cells. The standard deviation of the data reported was <10%. Regarding the results obtained with the combinations, some combinations were more effective than the single phytocannabinoid, as can be seen in Tables 2-5. FIG. 25 shows a plot of the results for the triple combinations. Since using 25 μM of each cannabinoid in the triple combination there was 100% cell mortality, a 12.5 μM dose was tested in the triple and quadruple combinations and a 6.25 μM dose was tested in the quadruple combination.

TABLE 2
Phytocannabinoids induce cytotoxicity in MM cell lines.

| | % VIABILITY ± S.D. | | |
|---|---|---|---|
| PHYTOCANNABINOID | U266 | RPMI | SKO |
| CBG (12.5 μM) | 97.4 ± 6.5 | 99.3 ± 8.2 | 105.4 ± 4.4 |
| CBG (25 μM) | 87.7 ± 5.8 | 69.1 ± 5.5 | 84.6 ± 5.5 |
| CBC (12.5 μM) | 106.2 ± 3.5 | 104.9 ± 3.6 | 96.5 ± 6.7 |
| CBC (25 μM) | 101.2 ± 7.2 | 98.3 ± 5.1 | 94.3 ± 6.3 |
| CBN (12.5 μM) | 89.6 ± 6.6 | 102.6 ± 7.4 | 89.2 ± 5.9 |
| CBN (25 μM) | 61.1 ± 4.8 | 81.1 ± 7.7 | 60.6 ± 3.8 |
| CBDV (12.5 μM) | 88.6 ± 7.2 | 70.2 ± 5.3 | 82.1 ± 5.2 |
| CBDV (25 μM) | 76.2 ± 6.5 | 40.2 ± 2.4 | 54.6 ± 4.4 |
| VEHICLE | 100 | 100 | 100 |

TABLE 3
Phytocannabinoid double combinations.

| | % VIABILITY | | |
|---|---|---|---|
| DOUBLE COMBINATION | U266 | RPMI | SKO |
| CBG 25 μM + CBN 12.5 μM | 75.5 ± 6.2 | 49.5 ± 3.0* | 64.4 ± 5.1* |
| CBG 25 μM + CBN 25 μM | 74.7 ± 6.3 | 15.3 ± 0.9* | 64.3 ± 5.4 |
| CBG 12.5 μM + CBN | 100 ± 8.8 | 88.8 ± 7.5 | 96.1 ± 8.4 |
| 12.5 μM | | | |
| CBG 12.5 μM + CBN | 100 ± 9.1 | 54.6 ± 4.6* | 92.7 ± 7.9 |
| 25 μM | | | |
| CBDV 25 μM + CBC | 68.2 ± 5.2 | 36.5 ± 2.7 | 59.8 ± 4.4 |
| 12.5 μM | | | |
| CBDV 25 μM + CBC 25 μM | 59.5 ± 4.7* | 27.7 ± 1.9* | 53.3 ± 4.1 |
| CBDV 12.5 μM + CBC | 93.6 ± 8.8 | 60 ± 5.3 | 76.1 ± 6.8 |
| 12.5 μM | | | |
| CBDV 12.5 μM + CBC | 94 ± 8.7 | 41.7 ± 3.5* | 73.8 ± 6.8 |
| 25 μM | | | |
| CBDV 25 μM + CBN | 57.4 ± 4.4 | 32.7 ± 2.0 | 50.2 ± 4.5 |
| 12.5 μM | | | |
| CBDV 25 μM + CBN 25 μM | 58.1 ± 4.9 | 16.8 ± 0.9* | 52.4 ± 4.3 |
| CBDV 12.5 μM + CBN | 93.2 ± 8.2 | 68.6 ± 5.5 | 78.3 ± 6.4 |
| 12.5 μM | | | |
| CBDV 12.5 μM + CBN | 84.1 ± 7.1 | 37.7 ± 2.2* | 73.1 ± 6.0 |
| 25 μM | | | |
| CBC 25 μM + CBN | 100 ± 8.8 | 31.6 ± 1.9* | 84.2 ± 7.0 |
| 12.5 μM | | | |
| CBC 25 μM + CBN 25 μM | 72.7 ± 6.5 | 16.8 ± 0.7* | 57.8 ± 4.6 |
| CBC 12.5 μM + CBN | 92 ± 8.1 | 87.5 ± 5.8 | 76.4 ± 6.3 |
| 12.5 μM | | | |
| CBC 12.5 μM + CBN | 94 ± 8.2 | 41.4 ± 3.3* | 71 ± 6.4 |
| 25 μM | | | |
| CBG 25 μM + CBDV | 61.6 ± 5.3 | 35.4 ± 2.2* | 55.2 ± 4.1* |
| 12.5 μM | | | |
| CBG 25 μM + CBDV 25 μM | 40.8 ± 3.1* | 15.8 ± 0.8* | 39.4 ± 2.9* |
| CBG 12.5 μM + CBDV | 86.2 ± 7.3 | 65.8 ± 4.8 | 74.5 ± 6.2 |
| 12.5 μM | | | |
| CBG 12.5 μM + CBDV | 57.8 ± 4.9* | 30.2 ± 1.9 | 55.6 ± 4.3 |
| 25 μM | | | |
| CBG 25 μM + CBC 25 μM | 51 ± 4.2* | 8.8 ± 0.6* | 45 ± 3.2* |
| CBG 25 μM + CBC | 57.3 ± 4.5* | 14.6 ± 0.9* | 60.7 ± 5.1* |
| 12.5 μM | | | |
| CBG 12.5 μM + CBC | 82.8 ± 7.3* | 45.4 ± 3.0* | 99.8 ± 8.4 |
| 25 μM | | | |
| CBG 12.5 μM + CBC | 86.1 ± 7.3* | 72.4 ± 6.1* | 89 ± 7.9* |
| 12.5 μM | | | |
| VEHICLE | 100 ± 9.3 | 100 ± 9.0 | 100 ± 8.7 |

*Combination more effective compared to sum of % cytotoxicity for individual compounds.

TABLE 4
Phytocannabinoid triple combinations.

| | % VIABILITY | | |
|---|---|---|---|
| TRIPLE COMBINATION | U266 | RPMI | SKO |
| CBG + CBC + CBDV | 84.5 ± 7.2 | 50.0 ± 4.1 | 67.8 ± 5.8 |
| (12.5 μM each one) | | | |
| CBG + CBC + CBN | 86.5 ± 7.3 | 69.5 ± 5.8 | 70.0 ± 6.1 |
| (12.5 μM each one) | | | |
| CBG + CBN + CBDV | 67.0 ± 5.6 | 34.6 ± 2.5 | 44.6 ± 3.5 |
| (12.5 μM each one) | | | |
| CBC + CBDV + CBN | 79.8 ± 6.9 | 43.8 ± 3.8 | 60.1 ± 5.2 |
| (12.5 μM each one) | | | |
| VEHICLE | 100 ± 9.1 | 100 ± 8.9 | 100 ± 8.6 |

TABLE 5
Phytocannabinoid quadruple combinations.

| | % VIABILITY | | |
|---|---|---|---|
| QUADRUPLE COMBINATION | U266 | RPMI | SKO |
| CBG + CBC + CBDV + CBN | 98.5 ± 8.9 | 93.3 ± 8.0 | 94.2 ± 8.3 |
| (6.25 μM each one) | | | |
| CBG + CBC + CBDV + CBN | 59.9 ± 4.7 | 25.2 ± 1.7 | 30.5 ± 2.2 |
| (12.5 μM each one) | | | |
| VEHICLE | 100 ± 8.8 | 100 ± 9.3 | 100 ± 9.1 |

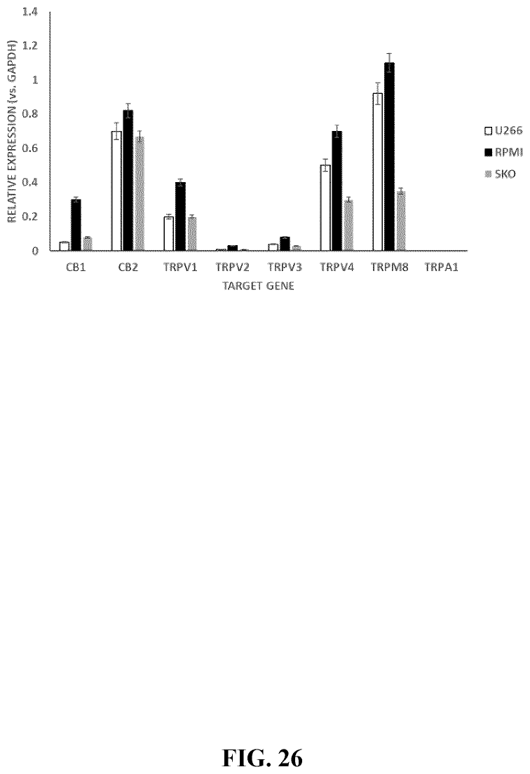

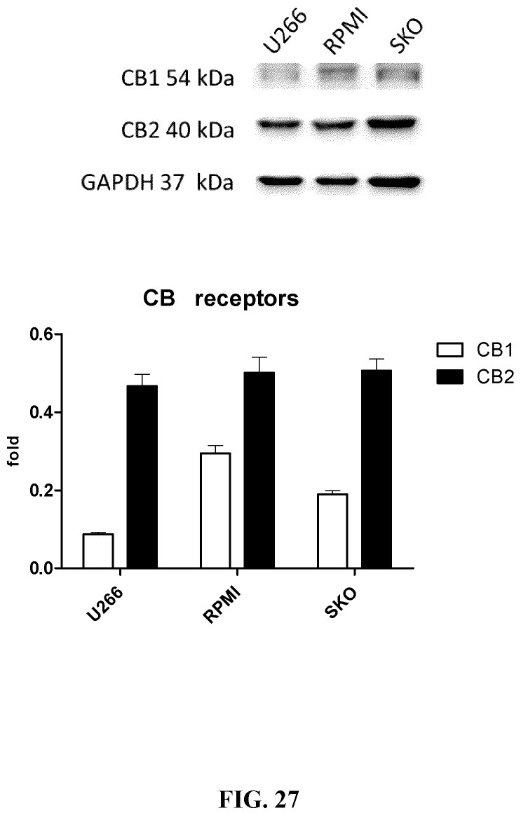

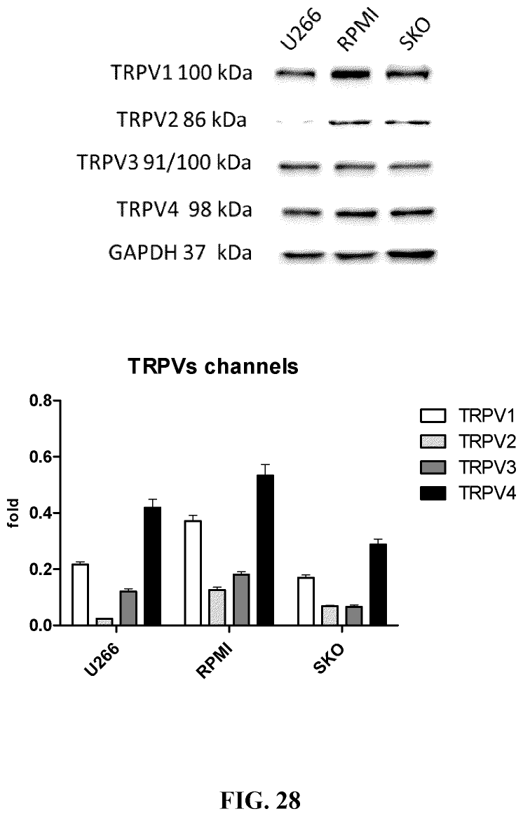

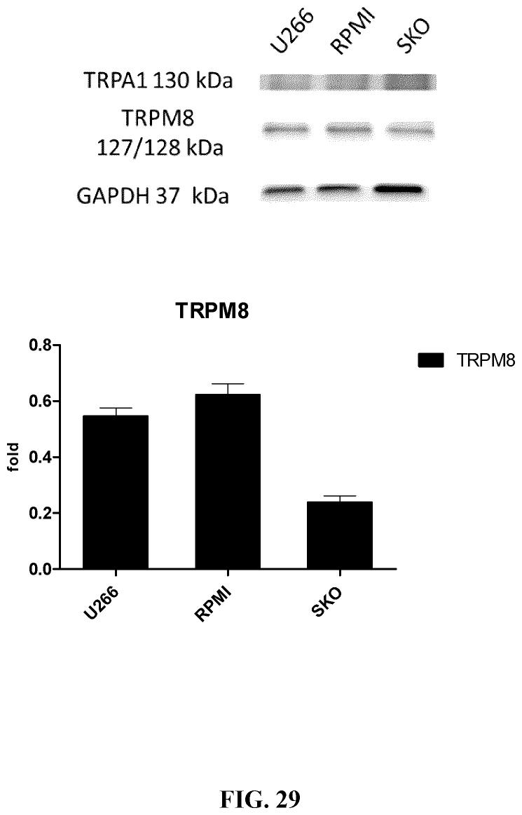

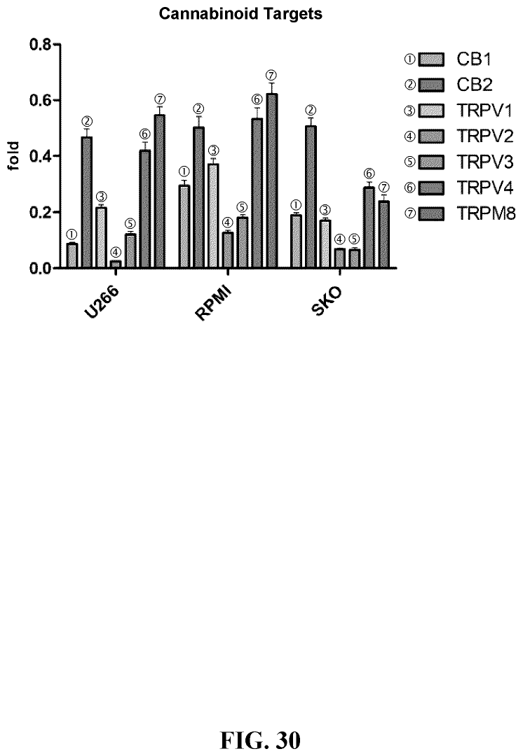

While not wishing to be limited by theory, cannabinoids can bind different receptors and may act as an agonist for some receptors and as an antagonist in others. Moreover, while not wishing to be limited by theory, cannabinoids can act also with a receptor-independent mechanism. Accordingly, the potential cannabinoid receptors were analyzed in the three cell lines (CB1, CB2, TRPV1, TRPV2, TRPV3, TRPV4, TRPM8 and TRPA1). While not wishing to be limited by theory, these analyses may provide some insight into the cannabinoids' effect on MM cell lines. FIG. 26 is a plot showing CB and TRP expression in the MM cell lines. FIGS. 27-30 show CB expression in MM cell lines (FIG. 27), TRPV expression in MM cell lines (FIG. 28), TRPM8 and TRPA1 expression in MM cell lines (FIG. 29) and cannabinoid target expression in MM cell lines (FIG. 30).

### Example 2: Effect of Phytocannabinoids and Combinations Thereof on HuOB Cell Line and on the Ability of MM Cell Lines to Migrate Toward HuOB

This study evaluated the cytotoxic activity of CBG, CBC, CBDV and CBN in human osteoblast cell line (HuOB) alone and in combination. In addition, the expression profile of the cannabinoid receptors evaluated in MM cell lines (Example 1), was determined in HuOB. Further, a potential cannabinoid therapy may desirably take into consideration the ability of MM cells to invade and metastasize bones. Accordingly, this study also evaluated the effects of the selected cannabinoids in reducing the ability of MM cell lines to migrate toward HuOB by an invasion assay in a MM cell line and HuOB co-culture.

I. Additional Materials and Methods

Cell culture: The HuOB, an immortalized human osteoblast cell line derived from femoral head (INS-CI-1005, Inscreeex GmbH, Germany) was grown in HuOB Maintenance Medium (INS-ME-1006).

Western blot analysis for cannabinoid ligands: The protocol was similar to that described hereinabove in Example 1 except lysates were from MM and HuOB cell lines.

Invasion assay: HuOB cells (1.5×10⁴) were plated on the surface of Matrigel in a BioCoat™ Matrigel® Invasion Chamber, 8.0 μm PET Membrane (Coming®, cod. 354480) and incubated for two days. After, U266 and RPMI (2.5×10⁴) were pre-stained with Calcein (1 μM) for two hours, then treated with CBG (12.5 μM), CBC (12.5 μM), CBN (12.5 μM) and CBDV (6.25 μM) and added in the chambers. MM cells with HuOB, untreated, were used as invasion positive control, MM cells without HuOB cells were used as negative control. After 24 hours, the number of MM cells that invaded the Matrigel were analysed via fluorescent microscope. Eight 10× sections images were counted for each chamber.

II. Results

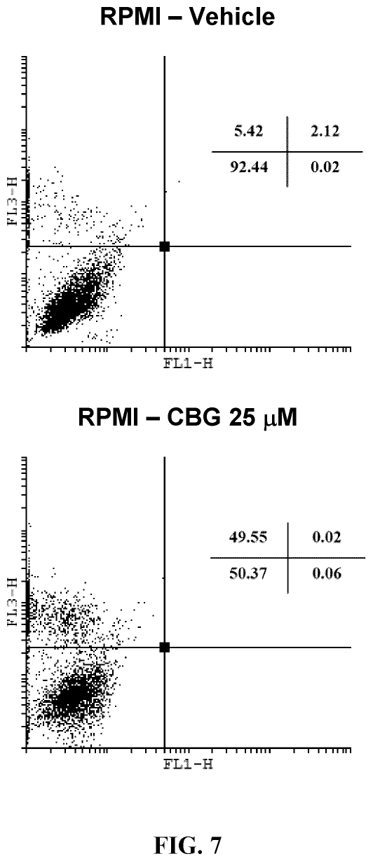

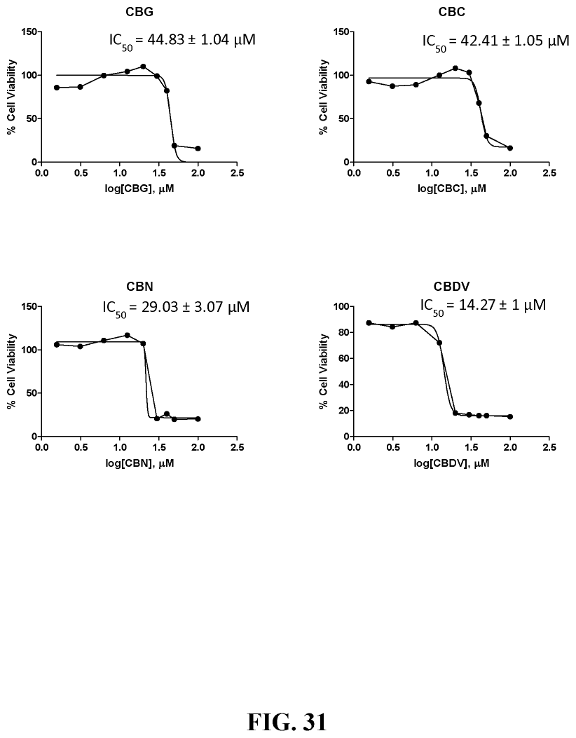

Phytocannabinoids induce cytotoxicity in the HuOB cell line: The effect of CBG, CBC, CBN and CBDV in reducing cell viability was evaluated at 72 hours, post daily administration, in the HuOB cell line. Cells were treated with different doses of CBG, CBC, CBN and CBDV (up to 100 μM) and percentage of cell viability was evaluated by the MTT assay. The results showed a dose dependent effect in the HuOB cell line for all of the phytocannabinoids (FIG. 31). IC₅₀ values, relative to daily administration, were obtained. The efficacy in reducing cell viability was generally highest for CBDV, followed by CBN, CBC and CBG. A comparative analysis of the IC₅₀ of the phytocannabinoids in HuOB and MM cell lines is provided in Table 6. FIG. 7 shows a comparative analysis of doses in the HuOB and MM cell lines.

TABLE 6
Comparative analysis of the IC₅₀ obtained
for HuOB and MM cell lines.

| | IC₅₀ (μM) ± S.D. | | | |
|---|---|---|---|---|
| | HuOB | U266 | SKO | RPMI |
| CBG | 44.83 ± 1.04 | 35.91 ± 2.2 | 35.02 ± 1.8 | 31.36 ± 1.9 |
| CBC | 42.41 ± 1.05 | 78.8 ± 3.4 | 69.7 ± 3.5 | 49.6 ± 2.1 |
| CBN | 29.03 ± 3.07 | 26.51 ± 1.8 | 28.5 ± 2.2 | 23.64 ± 0.9 |
| CBDV | 14.27 ± 1 | 26.71 ± 1.3 | 26.52 ± 2.1 | 21.68 ± 1.1 |

TABLE 7
Comparative analysis of doses in HuOB and MM cell lines.

| | % VIABILITY ± S.D. | | | |
|---|---|---|---|---|
| | HuOB | U266 | SKO | RPMI |
| CBG | 104.4 ± 8.2 | 97.4 ± 6.5 | 105.4 ± 4.4 | 99.3 ± 8.2 |
| (12.5 μM) | | | | |
| CBG (25 μM) | 96.4 ± 7.3 | 87.7 ± 5.8 | 84.6 ± 5.5 | 69.1 ± 5.5 |
| CBC | 100.1 ± 8.3 | 106.2 ± 3.5 | 96.5 ± 6.7 | 104.9 ± 3.6 |
| (12.5 μM) | | | | |
| CBC (25 μM) | 99.8 ± 8.1 | 101.2 ± 7.2 | 94.3 ± 6.3 | 98.3 ± 5.1 |
| CBN | 116.7 ± 7.7 | 89.6 ± 6.6 | 89.2 ± 5.9 | 102.6 ± 7.4 |
| (12.5 μM) | | | | |
| CBN (25 μM) | 81.7 ± 7.7 | 61.1 ± 4.8 | 60.6 ± 3.8 | 81.1 ± 7.7 |
| CBDV | 72.1 ± 5.8 | 88.6 ± 7.2 | 82.1 ± 5.2 | 70.2 ± 5.3 |
| (12.5 μM) | | | | |
| CBDV (25 μM) | 21.2 ± 1.1 | 76.2 ± 6.5 | 54.6 ± 4.4 | 40.2 ± 2.4 |

These experiments showed that doses of 6.25 μM and 12.5 μM were not cytoxic for HuOB. This was a necessary step before the study described in greater detail hereinbelow regarding the co-culture experiments with multiple myeloma cells and HuOB, to evaluate the invasion activity of MM cell lines toward bone tissue. The doses that were selected for the invasion assay were 12.5 μM for CBG, CBC and CBN, and 6.25 μM for CBDV (since more cytotoxic for HuOB).

Phytocannabinoid combinations in HuOB cell line viability: Before setting up the co-culture experiments with HuOB and MM cell lines, the less cytotoxic doses of phytocannabinoid combinations were evaluated in HuOB. By a MTT assay, a combination of CBG, CBC, CBN and CBDV at lower cytotoxic doses in pairs (Table 8), and triplets (Table 9) excluding CBDV, that showed high cytotoxicity for HuOB, was evaluated. The phytocannabinoids were administered daily for 72 hours, as in previous experiments at the described doses. Each combination was evaluated in six wells and in two separate experiments. The values in the tables are represented as % of cell viability compared to vehicle-treated cells. The standard deviation of the data reported was <10%. Regarding the results obtained with the combinations, some combinations were less cytotoxic (Table 8), in particular the combination CBG 12.5 μM+CBN 12.5 μM, that was selected as a double combination for the co-culture assay. Additionally, a triple combination with CBG, CBC and CBN at 12.5 μM for each phytocannabinoid, was performed (Table 9).

TABLE 8
Double combinations (12.5 and 25 μM) of phytocannabinoids in
HuOB and comparative analysis with data obtained in MM cell lines.

| | % VIABILITY ± S.D. | | | |
|---|---|---|---|---|
| DOUBLE COMBINATION | HuOB | U266 | RPMI | SKO |
| CBG 25 μM + CBN 12.5 μM | 65.1 ± 5.3 | 75.5 ± 6.2 | 49.5 ± 3.0* | 64.4 ± 5.1* |
| CBG 25 μM + CBN 25 μM | 22.2 ± 1.2 | 74.7 ± 6.3 | 15.3 ± 0.9* | 64.3 ± 5.4 |
| CBG 12.5 μM + CBN 12.5 μM | 104.5 ± 6.7 | 100 ± 8.8 | 88.8 ± 7.5 | 96.1 ± 8.4 |
| CBG 12.5 μM + CBN 25 μM | 23.6 ± 1.4 | 100 ± 9.1 | 54.6 ± 4.6* | 92.7 ± 7.9 |
| CBDV 25 μM + CBC 12.5 μM | 16.9 ± 0.5 | 68.2 ± 5.2 | 36.5 ± 2.7 | 59.8 ± 4.4 |
| CBDV 25 μM + CBC 25 μM | 17.4 ± 1.1 | 59.5 ± 4.7* | 27.7 ± 1.9* | 53.3 ± 4.1 |
| CBDV 12.5 μM + CBC 12.5 μM | 17.6 ± 0.9 | 93.6 ± 8.8 | 60 ± 5.3 | 76.1 ± 6.8 |
| CBDV 12.5 μM + CBC 25 μM | 15.9 ± 1.3 | 94 ± 8.7 | 41.7 ± 3.5* | 73.8 ± 6.8 |
| CBDV 25 μM + CBN 12.5 μM | 16.1 ± 1.1 | 57.4 ± 4.4 | 32.7 ± 2.0 | 50.2 ± 4.5 |
| CBDV 25 μM + CBN 25 μM | 16.7 ± 1.3 | 58.1 ± 4.9 | 16.8 ± 0.9* | 52.4 ± 4.3 |
| CBDV 12.5 μM + CBN 12.5 μM | 16.6 ± 0.8 | 93.2 ± 8.2 | 68.6 ± 5.5 | 78.3 ± 6.4 |
| CBDV 12.5 μM + CBN 25 μM | 16.1 ± 0.8 | 84.1 ± 7.1 | 37.7 ± 2.2* | 73.1 ± 6.0 |
| CBC 25 μM + CBN 12.5 μM | 27.6 ± 1.5 | 100 ± 8.8 | 31.6 ± 1.9* | 84.2 ± 7.0 |
| CBC 25 μM + CBN 25 μM | 19.4 ± 1.1 | 72.7 ± 6.5 | 16.8 ± 0.7* | 57.8 ± 4.6 |
| CBC 12.5 μM + CBN 12.5 μM | 90.5 ± 7.2 | 92 ± 8.1 | 87.5 ± 5.8 | 76.4 ± 6.3 |
| CBC 12.5 μM + CBN 25 μM | 19.1 ± 1.1 | 94 ± 8.2 | 41.4 ± 3.3* | 71 ± 6.4 |
| CBG 25 μM + CBDV 12.5 μM | 17.0 ± 0.5 | 61.6 ± 5.3 | 35.4 ± 2.2* | 55.2 ± 4.1* |
| CBG 25 μM + CBDV 25 μM | 17.0 ± 0.7 | 40.8 ± 3.1* | 15.8 ± 0.8* | 39.4 ± 2.9* |
| CBG 12.5 μM + CBDV 12.5 μM | 19.8 ± 1.1 | 86.2 ± 7.3 | 65.8 ± 4.8 | 74.5 ± 6.2 |
| CBG 12.5 μM + CBDV 25 μM | 16.7 ± 1.1 | 57.8 ± 4.9* | 30.2 ± 1.9 | 55.6 ± 4.3 |
| CBG 25 μM + CBC 25 μM | 29.3 ± 1.6 | 51 ± 4.2* | 8.8 ± 0.6* | 45 ± 3.2* |
| CBG 25 μM + CBC 12.5 μM | 76.9 ± 5.3 | 57.3 ± 4.5* | 14.6 ± 0.9* | 60.7 ± 5.1* |
| CBG 12.5 μM + CBC 25 μM | 83.8 ± 7.3 | 82.8 ± 7.3* | 45.4 ± 3.0* | 99.8 ± 8.4 |
| CBG 12.5 μM + CBC 12.5 μM | 105.3 ± 6.2 | 86.1 ± 7.3* | 72.4 ± 6.1* | 89 ± 7.9* |

*Combination more effective compared to sum of % cytotoxicity for individual compounds.

TABLE 9
Triple combination of phytocannabinoids CBG + CBC + CBN at 12.5 μM
in HuOB and comparative analysis with data obtained in MM cell lines.

| | % VIABILITY ± S.D. | | | |
|---|---|---|---|---|
| | HuOB | U266 | RPMI | SKO |
| CBG + CBC + CBN | 100.6 ± 5.8 | 86.5 ± 7.3 | 69.5 ± 5.8 | 70.0 ± 6.1 |
| (12.5 μM each one) | | | | |

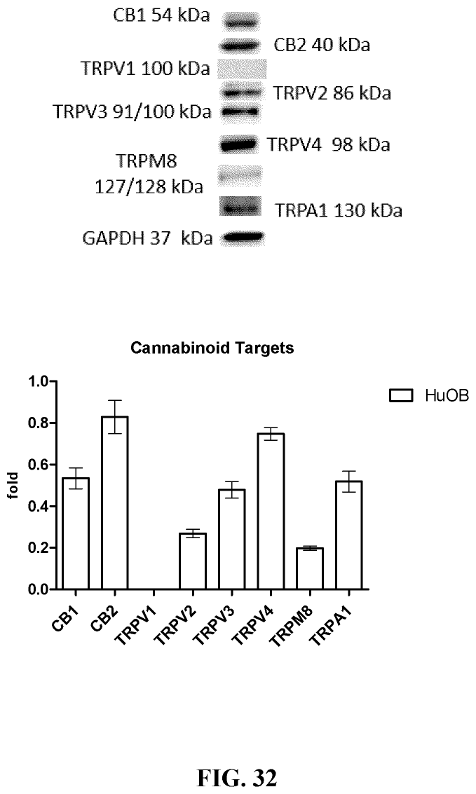

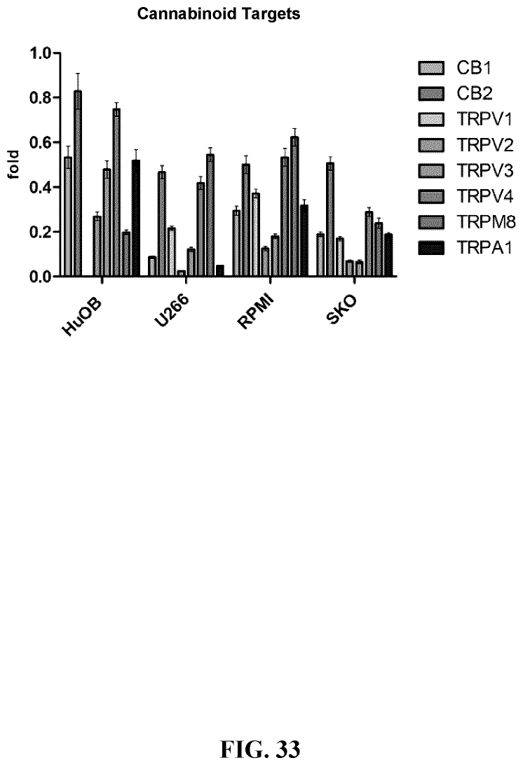

Cannabinoid receptor expression analysis in HuOB: The potential cannabinoid receptors (CB1, CB2, TRPV1, TRPV2, TRPV3, TRPV4, TRPM8 and TRPA1) were detected in HuOB by Western Blot analysis. As shown in FIG. 32, HuOB expressed CB2 at a higher level, while no expression of TRPV1 was detected. Further, a comparative analysis of cannabinoid receptor expression is shown in FIG. 33. The data evidenced that the major cannabinoid receptors expressed in HuOB compared to MM cell lines were CB2 and TRPV4.

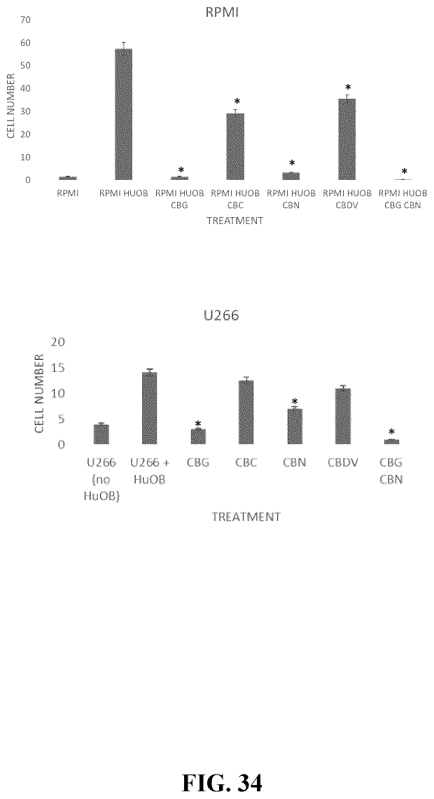

Invasion assay in co-culture of HuOB and MM cell lines: This study evaluated the effect of phytocannabinoids in reducing the invasion of MM cell lines toward HuOB. HuOB were plated on the surface of Matrigel in the BioCoat™ Matrigel® Invasion Chamber, 8.0 μm PET Membrane (Corning®, cod. 354480) and incubated for two days. After, U266 or RPMI were pre-stained with Calcein (10 μM) for two hours, then treated with CBG (25 μM), CBC (25 μM), CBN (25 μM) and CBDV (12.5 μM) and added in the chambers. MM cells with HuOB, untreated, were used as an invasion positive control, MM cells without HuOB cells were used as a negative control. After 24 hours, the number of MM cells that invaded the Matrigel were analysed via fluorescent microscope. Eight 10× sections images were counted for each chamber. The results showed no invasion of MM cells in negative control (MM cells without HuOB), while a higher number of MM cells was counted in positive control wells (MM cells in co-culture with HuOB). Regarding phytocannabinoids, the results showed that in both MM cell lines, the phytocannabinoids exerted an inhibitory effect on MM cell invasion. A higher activity was observed for CBG and CBN, followed by CBC and CBDV. The combination of CBG plus CBN showed nearly complete inhibition of MM cell invasion (FIGS. 34 and 35; Tables 10 and 11).

TABLE 10

| TREATMENT | AVERAGE OF CELLS/square |
|---|---|
| RPMI (no HuOB) | 1.6 ± 0.1 |
| RPMI + HuOB | 57.3 ± 3.4 |
| CBG | 1.6 ± 0.2 |
| CBC | 29.3 ± 1.7 |
| CBN | 3.3 ± 0.1 |
| CBDV | 35.6 ± 2.4 |
| CBG + CBN | 0.3 ± 0.1 |

TABLE 11

| TREATMENT U266 | AVERAGE OF CELLS/square |
|---|---|
| U266 (no HuOB) | 4.1 ± 0.3 |
| U266 + HuOB | 14.2 ± 0.9 |
| CBG | 3.2 ± 0.2 |
| CBC | 12.5 ± 1.1 |
| CBN | 7.0 ± 0.2 |
| CBDV | 11.2 ± 1.1 |
| CBG + CBN | 2.3 ± 0.1 |

While the disclosure has been described with reference to what are presently considered to be the preferred examples, it is to be understood that the disclosure is not limited to the disclosed examples. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE DESCRIPTION

¹ Kyle R A, Gertz M A, Witzig T E, et al. “Review of 1027 patients with newly diagnosed multiple myeloma” Mayo Clin Proc. 2003; 78(1):21-33.
² Nakashima T, Hayashi M, Fukunaga T, et al. “Evidence for osteocyte regulation of bone homeostasis through RANKL expression” Nat Med. 2011; 17(10):1231-1234.
³ Morelli et al., “The effects of cannabidiol and its synergism with bortezomib in multiple myeloma cell lines. A role for transient receptor potential vanilloid type-2” Int. J. Cancer 2014, 134:11, 2534-46.
⁴ Nabissi et al., “Cannabinoids synergize with carfilzomib, reducing multiple myeloma cells viability and migration” Oncotarget 2016, 7:47, 77543-77557.

What is claimed is:

1. A use of an effective amount of at least one phytocannabinoid for treatment of a plasma cell neoplasm in a subject in need thereof,
with the proviso that the at least one phytocannabinoid does not comprise cannabidiol or tetrahydrocannabinol.

2. A use of an effective amount of at least one phytocannabinoid for preparation of a medicament for treatment of a plasma cell neoplasm in a subject in need thereof,
with the proviso that the at least one phytocannabinoid does not comprise cannabidiol or tetrahydrocannabinol.

3. The use of claim 1 or 2, wherein the at least one phytocannabinoid comprises cannabidivarin, cannabinol, cannabigerol, cannabichromene or combinations thereof.

4. The use of claim 1 or 2, wherein the at least one phytocannabinoid is cannabidivarin, cannabinol, cannabigerol, cannabichromene or combinations thereof.

5. The use of claim 1 or 2, wherein the at least one phytocannabinoid is cannabidivarin.

6. The use of claim 1 or 2, wherein the at least one phytocannabinoid is cannabinol.

7. The use of claim 1 or 2, wherein the at least one phytocannabinoid is cannabichromene.

8. The use of claim 1 or 2, wherein the at least one phytocannabinoid is cannabigerol.

9. The use of claim 1 or 2, wherein the at least one phytocannabinoid is a combination of cannabigerol and cannabichromene.

10. The use of claim 3, wherein the at least one phytocannabinoid is a combination comprising cannabinol.

11. The use of claim 10, wherein the combination further consists of cannabigerol.

12. The use of claim 10, wherein the combination further consists of cannabichromene.

13. The use of claim 10, wherein the combination further consists of cannabidivarin.

14. The use of any one of claims 1 to 13, wherein the plasma cell neoplasm is multiple myeloma.

15. The use of claim 14, wherein the subject has myeloma bone disease and the at least one phytocannabinoid is further for treatment of the myeloma bone disease.

16. A use of an effective amount of at least one phytocannabinoid for treatment of myeloma bone disease in a subject in need thereof,
with the proviso that the at least one phytocannabinoid does not comprise cannabidiol or tetrahydrocannabinol.

17. A use of an effective amount of at least one phytocannabinoid for preparation of a medicament for treatment of myeloma bone disease in a subject in need thereof,
with the proviso that the at least one phytocannabinoid does not comprise cannabidiol or tetrahydrocannabinol.

18. The use of claim 16 or 17, wherein the at least one phytocannabinoid is cannabinol.

19. The use of claim 16 or 17, wherein the at least one phytocannabinoid is cannabigerol.

20. The use of claim 16 or 17, wherein the at least one phytocannabinoid is a combination of cannabinol and cannabigerol.

21. The use of any one of claims 1 to 20, wherein the at least one phytocannabinoid is administered in combination with at least one other anticancer treatment.

22. The use of claim 21, wherein the other anticancer treatment is a proteasome inhibitor, an immunomodulator or combinations thereof.

23. The use of any one of claims 1 to 22, wherein the subject is a human.

24. The use of any one of claims 1 to 23, wherein the at least one phytocannabinoid is administered to the subject in the form of a pharmaceutical composition comprising the at least one phytocannabinoid and a pharmaceutically acceptable carrier.

25. The use of any one of claims 1 to 24, wherein the at least one phytocannabinoid is administered orally to the subject.

26. The use of claim 25, wherein the at least one phytocannabinoid is administered to the subject in the form of a pharmaceutical composition comprising the at least one phytocannabinoid and a pharmaceutically acceptable oil.

27. The use of claim 25, wherein the at least one phytocannabinoid is administered to the subject in the form of a gastro-resistant capsule comprising the at least one phytocannabinoid.

28. The use of any one of claims 1 to 24, wherein the at least one phytocannabinoid is administered to the subject by inhalation via vaporization.

29. The use of any one of claims 1 to 28, wherein the at least one phytocannabinoid is administered to the subject once daily.
